(19) United States
(12) Patent Application Publication
(10) Pub. No.: US 2015/0369803 A1
(43) Pub. Date: Dec. 24, 2015

(54) LOW FLUORESCENCE UTENSILS

(71) Applicants: Dina KATSIR, Lehavim (IL); Zvi FINKELSTEIN, Asseret (IL)

(72) Inventors: Dina KATSIR, Lehavim (IL); Zvi FINKELSTEIN, Asseret (IL)

(21) Appl. No.: 14/764,578

(22) PCT Filed: Jan. 30, 2014
(86) PCT No.: PCT/IL2014/050105

§ 371 Date: Jul. 30, 2015

Related U.S. Application Data
(60) Provisional application No. 61/758,871, filed on Jan. 31, 2013.

(51) Int. Cl. G01N 33/543; C23C 16/56; G01N 21/64; C23C 16/40

(52) U.S. Cl. CPC G01N 33/54393; C23C 16/403; C23C 16/405; C23C 16/401; C23C 16/407; C23C 16/56; G01N 21/645; G01N 2021/6482

(57) ABSTRACT

Method of reducing background fluorescence and corresponding utensils are provided. Utensils such as those used for fluorescence measurements in many fields are coated by a coating comprising porous fractal ceramic layer(s). The ceramic layer is produced by reactive vapor deposition and comprises oxides of aluminum, titanium, tantalum, niobium, zirconium, silicon, thorium, cadmium or tungsten. The coating is configured to exhibit hemispherical reflectance of less than 2% over a range of wavelengths ranging from 0.1-10 μm. The coating achieves high resolution by applying fine deposition and ablation patterns and due to its porousness, and is non-toxic and non-outgassing.

### BACKGROUND OF THE INVENTION

1. Technical Field

The present invention relates to the field of utensils for laboratory analysis, and more particularly, to utensils for fluorescence measurements.

2. Discussion of Related Art

Fluorescence measurements are wide-spread in the fields of medicine, biology and chemistry as well in other fields.

### SUMMARY OF THE INVENTION

One aspect of the present invention provides a method comprising reducing background fluorescence in a utensil by producing at least an imaged region of the utensil to have a coating comprising at least one porous fractal ceramic layer produced by reactive vapor deposition and comprising at least one oxide of: aluminum, titanium, tantalum, niobium, zirconium, silicon, thorium, cadmium and tungsten, wherein the coating is configured to exhibit hemispherical reflectance of less than 2% over a range of wavelengths ranging from 0.1-10 μm. In certain embodiments, the coating achieves high resolution by applying fine deposition and ablation patterns and due to its porousness, and is non-toxic and non-outgassing.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of Example, to the accompanying drawings in which like numerals designate corresponding elements or sections throughout.

In the accompanying drawings:

FIGS. 1A-1F are high level schematic illustrations of utensils having reduced background fluorescence at imaged regions, according to some embodiments of the invention.

FIGS. 2A-2G are high level schematic illustrations of coatings that reduce background fluorescence at imaged regions of utensils, according to some embodiments of the invention.

FIGS. 3A-3E are illustrative microscopic images of a porous fractal ceramic layer, according to some embodiments of the invention.

FIGS. 4A-4D illustrate the porosity and fractal structure of the coatings, according to some embodiments of the invention.

FIGS. 5A-5G present fluorescence and reflection measurements of the coatings, according to some embodiments of the invention.

FIG. 6A-6C present the setting and measurements related to laser induced damage of the coating, according to some embodiments of the invention.

FIG. 7 illustrates a scheme of an in-line process carried out inside a vacuum chamber, which enables production of coated foil in mass production volumes, according to some embodiments of the invention.

FIG. 8 is a high level schematic flowchart illustrating a method, according to some embodiments of the invention.

### DETAILED DESCRIPTION OF THE INVENTION

Prior to the detailed description being set forth, it may be helpful to set forth definitions of certain terms that will be used hereinafter.

The terms “utensil” or “apparatus” as used in this application refer to any article used to perform fluorescence measurements, for example biological, medical or chemical fluorescence measurements. As non-limiting examples, the term “utensil” may be understood as referring to any of: a multi well plate, a foil, an embossed foil, a continuous array tape, a foil metal insert, a lab-on-a-foil, DNA or protein microarrays, a glass slide, a flow cell, a microscope pad, microbeads, membranes, biochips and other life science products used in florescence measurements.

The term “ceramic” as used in this application refers to is an inorganic, nonmetallic solid having a crystalline structure, a partly crystalline structure or an amorphous structure. In a non-limiting example, The term “ceramic” as used in this application refers to an inorganic solid consisting one or more metal and/or their corresponding oxides.

The term “dielectric” as used in this application refers to electrically insulating materials which are polarized by applied electric fields. In a non-limiting example, most ceramics are dielectrics.

The terms “metal” and “metals” include single metallic elements and admixtures and alloys thereof, unless the context specifically indicates the contrary.

“Valve metal” refers to a metal which, when oxidized, allows current to pass if used as cathode but opposes the flow of current when used as anode. Examples of valve metals include magnesium, thorium, cadmium, tungsten, tin, iron, silver, silicon, tantalum, titanium, aluminum, zirconium and niobium. In other embodiments, the valve metal is selected from the group consisting of magnesium, thorium, cadmium, tungsten, tin, iron, silver, silicon, tantalum, titanium, aluminum, zirconium and niobium.

The term “vacuum deposition” includes known vacuum deposition techniques, including thermal resistive evaporation, electron beam evaporation, electric arc deposition, laser deposition and sputtering.

“Polypeptide” refers to a polymer in which the monomers are amino acid residues which are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced. Antibodies are just one of many examples of polypeptides.

“Protein” refers to a biological molecule expressed by a gene and comprised of amino acids.

As used herein, “comprising” is to be interpreted as specifying the presence of the stated features, integers, steps, or components as referred to, but does not preclude the presence or addition of one or more features, integers, steps, or components, or groups thereof. Thus, for example, an apparatus comprising a given coating may contain additional components. Additionally, the term “comprising” is intended to include, as separate embodiments, embodiments encompassed by the terms “consisting essentially of” and “consisting of”. The phrase “consisting essentially of” limits the scope of a claim to the recited materials or steps, either alone or in combination with additional materials or steps that do not materially affect the basic and novel characteristics of the claimed invention.

Fluorescence is the emission of electromagnetic radiation, especially of visible light. Induced fluorescence is fluorescence that is stimulated in a substance by the absorption of incident light or other electromagnetic radiation, typically persisting only as long as the stimulating radiation is continued or for a defined interval thereafter. Fluorescence is a form of luminescence. In most cases, the emitted light has a longer wavelength, and therefore lower energy, than the absorbed radiation. However, when the absorbed electromagnetic radiation is intense, it is possible for one electron to absorb two photons; this two-photon absorption can lead to emission of radiation having a shorter wavelength than the absorbed radiation. The emitted radiation may also be of the same wavelength as the absorbed radiation, termed “resonance fluorescence”.

Reflectance, by contrast, is the change in direction of a wavefront at an interface between two different media, so that the wavefront returns into the medium from which it originated. Reflection of light may be either specular (mirror-like) or diffuse (retaining the energy, but losing the image) depending on the nature of the interface. Furthermore, the phase of the reflected wave may be either retained or inverted. Reflection may be expressed as the ratio of reflected radiant flux to incident radiant flux, or a the reflectance factor, namely the ratio of the flux reflected by the sample in a given direction to the flux reflected by a perfect reflecting diffuser (prd) identically irradiated and viewed.

The microplate/microwell plate/microtiter plate has become a standard tool in analytical research and clinical diagnostic testing laboratories. Microplates are widely used for gene discovery, drug screening, protein analysis, assay development and validation, biomolecule concentration measurement, and quality control and manufacturing processes; they are also used for high-throughput screening applications, DNA-quantifications and quality control. Their advantages include small sample/reagent volumes and multiparallel, on-plate analysis. The most common format is the 96-well plates, with 6, 12, 24, 48, 384, 1536 and 3456 well plates also available.

Microplates are available in various types, including different well geometries (flat bottom, round bottom, V-shape or deep wells are available); colored microplates for different optical readouts (e.g. black for fluorescence; white for luminescence); solid bottom wells or clear bottom wells for top- or bottom-reading fluorescence, respectively; different plastics (polystyrene, polypropylene, etc.); various coatings (e.g., reduction of non-specific binding, tissue-culture treated, etc.).

The most common used polymer is polystyrene, used for most optical detection microplates. It can be colored white by the addition of titanium dioxide for optical absorbance or luminescence detection or black by the addition of carbon for fluorescent biological assays. Non-transparent especially solid black polystryrene plates are designed to reduce well-to-well crosstalk and background. Black plates are available with clear bottoms for use in cell-based assays and microscopy applications, and allow top or bottom reading capabilities.

Polypropylene is used for the construction of plates subject to wide changes in temperature, such as storage at −80° C. and thermal cycling. The polypropylene material is also highly resistant to many commonly used solvents. Polypropylene microplates are ideal for compound storage or assays that require high resistance to solvents including DMSO and ethanol. It has excellent properties for the long-term storage of novel chemical compounds. Black polypropylene microplates can be used for fluorescent assays and reduce nonspecific binding problems observed with polystyrene plates.

Polycarbonate is inexpensive and easy to mold and is used for disposable microplates for the polymerase chain reaction (PCR) method of DNA amplification. Cyclo-olefins are now being used as the material for microplates which transmit ultraviolet light for use in newly-developed assays.

Flexible vinyl (PVC) microplates are economical, non-sterile general assay 96 well plates. Due to their flexible nature, these microplates are not compatible with automation.

The most common manufacturing process is injection die molding, used for polystyrene, polypropylene and cyclo-olefin. Vacuum forming can be used with softer plastics such as polycarbonate.

Composite microplates, such as filter plates and SPE plates and even some advanced PCR plate designs use multiple components which are molded separately and later assembled into a finished product.

Polymers such as polystyrene and polypropylene usually are untreated and hydrophobic to varying degrees, either repelling water or being non-wettable. Specialized surface treatments have been developed to produce surfaces for different applications.

A variety of fluorescence-based assays are performed in multi-well plates. In addition to standard fluorescence-based assays, these assays include fluorescence-resonance energy transfer (FRET), including more specialized assays such as DELFIA assays, which are TR-FRET assays using a Europium chelate, Terbium chelate, and/or Samarium chelate as the donor fluorophore. DELFIA assays utilize time-resolved FRET technology (TR-FRET).

Due to limitations in sample volumes and the need to reduce time and costs, new technologies have been developed. These include multiplex assay methodologies, which enable the researcher to test multiple analytes at one time within a single well, in contrast to the conventional ELISA arrays. Multiplexing offers several distinct advantages over single-plex assays, for example: A multiplex assay enables researchers to extract more data from a given amount of sample much more rapidly than single-plex assays; The typical ELISA requires 100-200 μl of sample per well for one protein. By comparison, typical multiplex assays often require less than 50 μl per reaction; Multiplex assays are also faster (because multiple analytes are being analyzed in parallel), more amenable to automation, and more frugal with reagents than individual single-plex assays—which translates into cost savings.

Membranes for separation of biological materials are known in the art, and are useful for various purposes.

Any of the above may be considered as the utensil described below.

With specific reference now to the drawings in detail, it is stressed that the particulars shown are by way of Example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

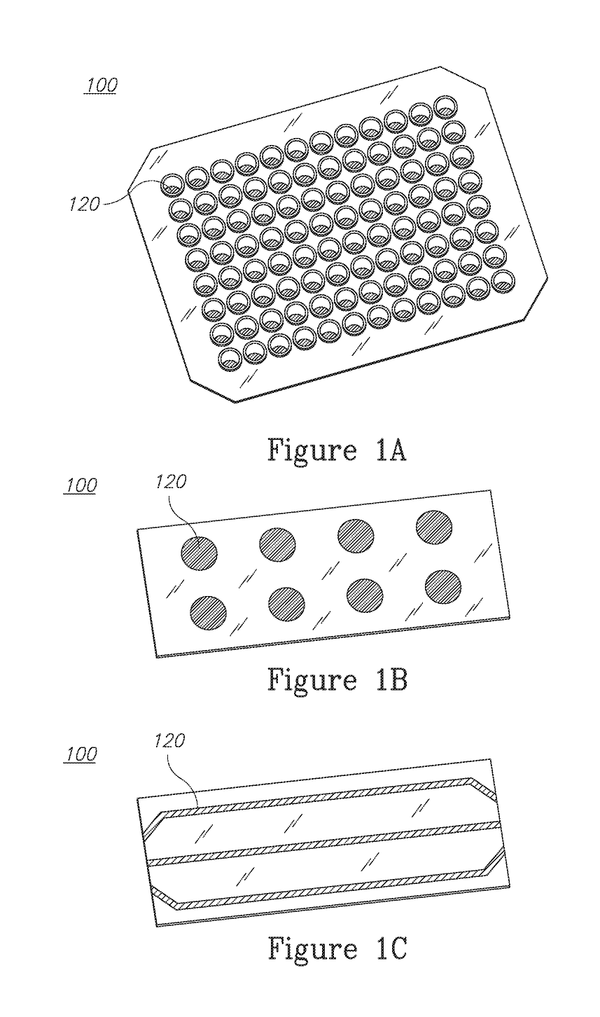

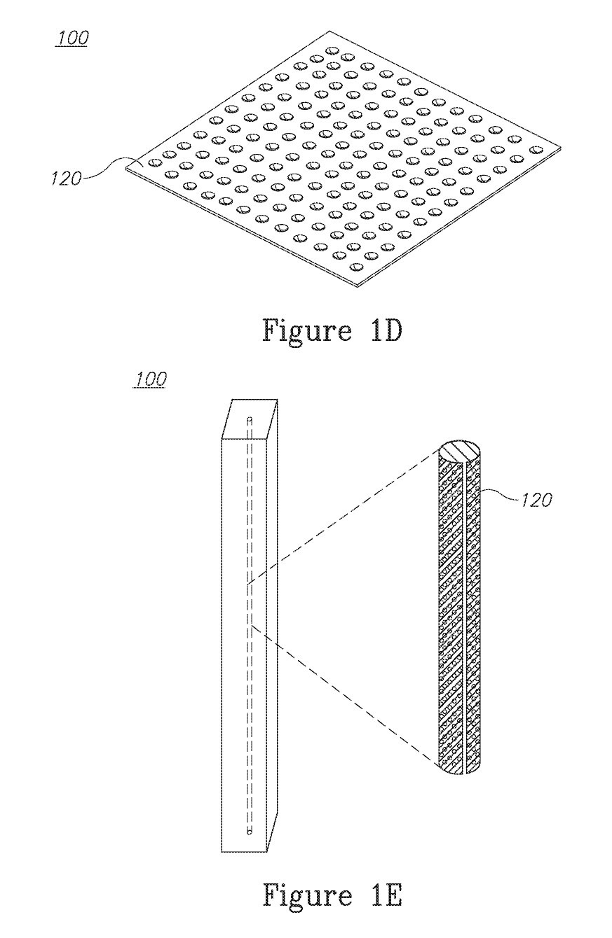

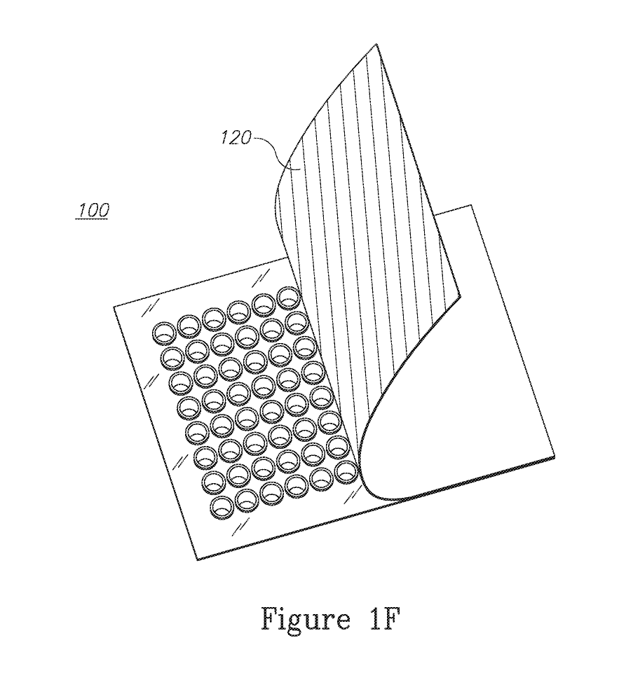

FIGS. 1A-1F are high level schematic illustrations of utensils 100 having reduced background fluorescence at imaged regions 110, according to some embodiments of the invention. As non-limiting applicative Examples, FIG. 1A illustrates a multi-well plate, FIGS. 1B and 1C illustrate glass slide having different designs, FIG. 1D illustrates an embossed foil, FIG. 1E illustrates a flow cell, FIG. 1F illustrates a multi well plate having a coated foil as the bottom layer.

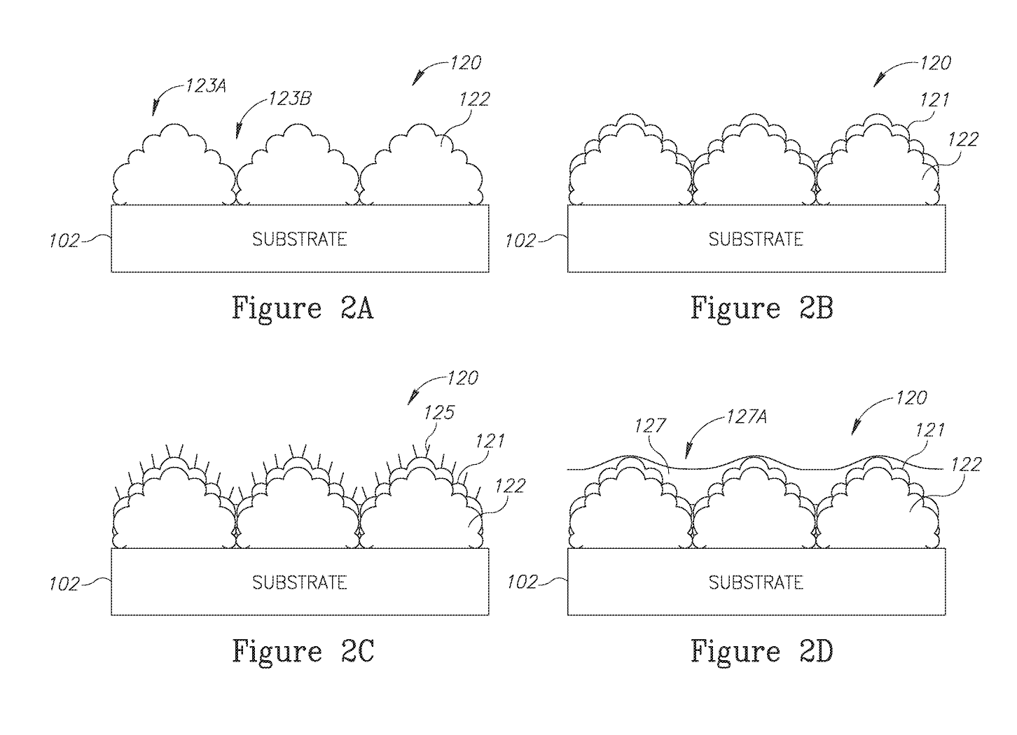

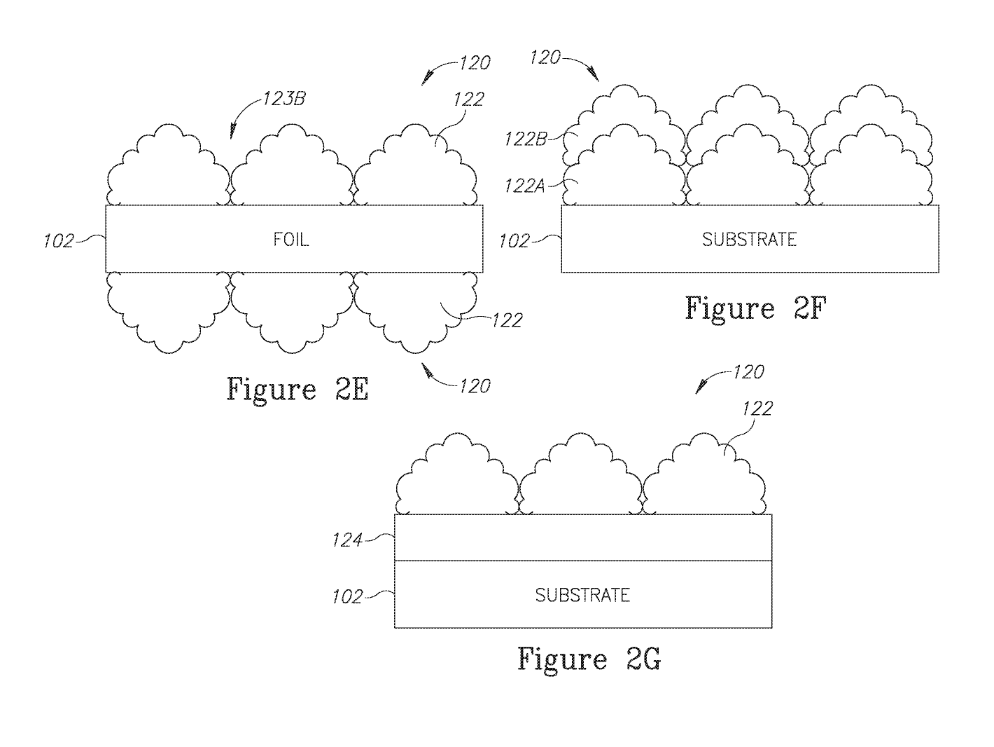

FIGS. 2A-2G are high level schematic illustrations of coatings 120 that reduce background fluorescence at imaged regions 110 of utensils 100, according to some embodiments of the invention. As non-limiting Examples, FIG. 2A illustrates coating 120 having a porous fractal ceramic layer 122 deposited upon a supporting substrate 102 (e.g., a metal layer), according to some embodiments of the invention. Support 102 may be made of any material, may be part of utensil 100 or may be an additional material (e.g., an attached foil). See Example 7 below for a quantification of the porosity of ceramic layer 122. Coating 120 may be deposited in one or more steps upon a substrate, such as a support 102 which may be part of utensil 100 or be an additional support. Coating 120 itself may be essentially ceramic, essentially metallic or a mix of ceramic and metallic material.

FIG. 2A schematically illustrates the fractal nature of ceramic layer 122, which simultaneously increases the surface area of layer 122 (arrow 123A) and creates pores through layer 122 (arrow 123B). The inventors have discovered that the fractal structure is controllable in a way that allow close control on the optical characteristics of coating 120, such as absorption and fluorescence at various wavelength ranges, as well as close control on the physical, chemical and biological binding characteristics of coating 120, such as its hydrophobicity and hydrophilicity, its levels of outgassing and toxicity (see below), and the coatings capacity to bind biological molecules. The parameters of the fractality of layer 122 may be controlled during the deposition process and by controlling the parameters thereof, and hence may be determined with respect to specific applications, as explained above.

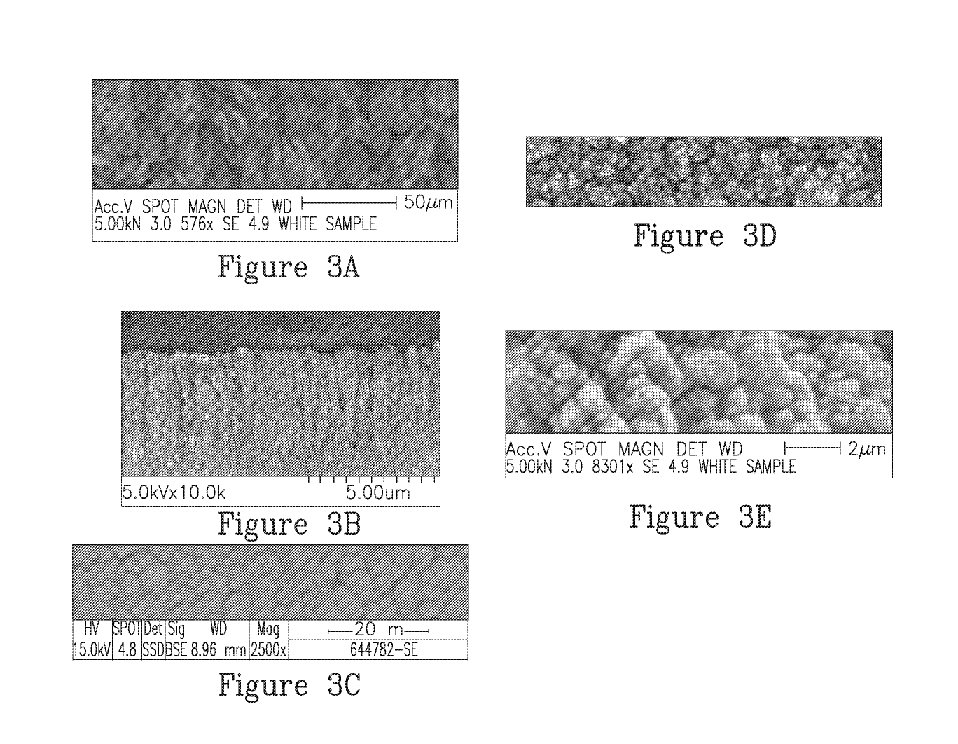

FIGS. 3A-3E are illustrative microscopic images of porous fractal ceramic layer 122, according to some embodiments of the invention. FIG. 3B is a cross of ceramic layer 122 while FIGS. 3A and 3C-3E are top views of ceramic layer 122. FIGS. 3A-3E illustrate the following types of fractal structure of layer 122: dendrite (FIG. 3A), coral-like (FIG. 3B) and cauliflower-like (FIGS. 3C-3E).

FIG. 2B schematically illustrates an over-coating layer 121 which may be deposited upon ceramic layer 122 and be part of coating 120. Over-coating layer 121 may be selected to control the coating's surface energy and determine its grade of hydrophobicity or hydrophilicity. Over-coating layer 121 may be selected to enhance binding by bridging between ceramic layer 122 and the relevant molecules that are to be bound to coating 120. Over-coating layer 121 may be applied after the production of coating 120, e.g. upon a coated foil. In certain embodiments, over-coating layer 121 may be substantially thinner than ceramic layer 122. For example, while ceramic layer 122 may be tens of μm thick (e.g., 10-30 μm) or several μm thick (e.g., 0.3-10 μm), over-coating layer 121 may be one or two orders of magnitude thinner (e.g., 0.1-2 μm). Over-coating layer 121 may be applied to one or both sides of a coated element. Over-coating layer 121 may be itself multi-layered. Over-coating layer 121 may comprise a polymer such as epoxy, SiO₂, or other relevant materials.

FIG. 2C schematically illustrates bound molecules 125 attached to over-coating layer 121. Molecules 125 may be selected to enable various functions of utensil 100 and may comprise any of the various molecules exemplified below. Molecules 125 may be bound directly to ceramic layer 122. Molecules 125 may comprise multiple types of molecules, and possible multi-layered bounding (e.g., several antigens). Molecules 125 may be biomolecules 125 bound upon porous fractal ceramic layer 122. Biomolecules 125 may be applied as a layer or in any pattern and in any amount specified by requirements.

FIG. 2D schematically illustrates a layer 127 attached to over-coating layer 121. Layer 127 may comprise any liquid of any composition. For example, Layer 127 may comprise marking material such as a pigment solution. Layer 127 may form thicker film regions at pores 123B in ceramic layer 122 creating thereby a localization effect, which may be utilize to enhance the detection efficiency of, e.g., pigments in layer 127. Thus, the fractal form of coating 120 may be used to concentrate required signals and achieve higher sensitivities for test for which utensil 100 is used. Layer 127 may be bound directly onto ceramic layer 122, and on one or both sides of utensil 100.

FIG. 2E schematically illustrates coating 120 on both sides of foil 100 (made of substrate 102). Ceramic layers 122 may be similar in their characteristics or may differ from each other (e.g., in thickness, porosity, composition etc.) and be useful for different purposes. One or both sides of foil 100 may further comprise over-coating layer 121 and/or bound molecules 125 and/or layer 127 described above.

In certain embodiments, foil 100 with coating 120 on one or both sides may be etched through and thus comprise pores. In certain embodiments, etching foil 100 may be used to produce a membrane structure, or a stack of such foils may be used as a membrane element. In certain embodiments, coating 120 may be applied to a porous foil or a membrane having a high ratio between the area of the pores and the area of the material left between the pores, e.g. a pore/material ratio larger than 1. In such embodiments, coating 120 may be applied onto he inner sides of the pores as well as to the foil or membrane surface.

FIG. 2F schematically illustrates coating 120 having multiple ceramic layers 122A, 122B which have similar or different characteristics (composition, spatial structure, porosity etc.). Multiple layers may provide required coating characteristics with respect to parameters such as binding efficiency, surface energy, porosity, toxicity, outgassing, optical parameters and fluorescence.

FIG. 2G schematically illustrates coating 120 having ceramic layers 122 deposited upon a metal layer 124 which is deposited or attached to substrate 102. Any combination of the layers described above may be used to meet specific requirements.

Utensil 100 has coating 120 comprising at least one porous fractal ceramic layer 122 deposited upon at least one metal layer 124. Ceramic layer 122 may be produced by reactive vapor deposition and comprise at least one oxide of: aluminum, titanium, tantalum, niobium, zirconium, silicon, thorium, cadmium and tungsten. Coating 120 may be configured to exhibit hemispherical reflectance of less than 2% over a range of wavelengths ranging from 0.1-10 μm to reduce background fluorescence of at least one imaged region 110 of utensil 100. See Examples 1 and 2 below for exemplary preparation of coating 120, according to some embodiments of the invention.

In certain embodiments, utensil 100 may be a multi-well plate with coated well bottoms as the imaged region. Utensil 100 may be a glass slide with patterned coated imaged regions. For Example, coating 120 in any of the utensil embodiments may have a specified pattern designed according to a microscopy target. The coating pattern may exhibit a specified hydrophobic/hydrophilic character. Utensil 100 may be a flow cell with at least partially coated flow channels.

In certain embodiments, utensil 100 may be a foil having the coating. Utensil 100 may be an embossed foil having coated indentations as imaged regions. The foil may have coating 120 on both of its sides. Utensil 100 may comprise a foil with coating 120, attached to the imaged regions of utensil 100. In certain embodiments, utensil 100 may be a membrane having coating 120 on both sides. See Examples 3-6 below for evidence for the low fluorescence of coating 120.

### Types of Utensils

In certain embodiments, utensil 100 may comprise a multi-well plate (FIG. 1A), namely an apparatus comprising a frame, which contains within it a plurality of containers suitable for containing a liquid (“wells”). Exemplary, non-limiting embodiments of multi-well plates are plates intended for conducting laboratory assays and plates intended for tissue culture. In certain, more specific embodiments, the plates are suitable for conducting laboratory assays that comprise adherent cells; the plate is thus sterilizable, if intended for assays lasting more than a few hours, and the inner surface of the wells contains a substrate for cell adherence and does not elute significant amounts of one or more cytotoxic substances when aqueous solution is incubated in its wells. In other embodiments, the plates are suitable for conducting laboratory assays in suspension cell culture; the plate is sterilizable, if intended for assays lasting more than a few hours, and the inner surface of the wells need not contain a substrate for cell adherence, but in any case does not elute significant amounts of one or more cytotoxic substances when aqueous solution is incubated in its wells. “Significant amounts” in this context refers to an amount of cytotoxic substances that adversely affects cellular viability to an appreciable extent—for Example by decreasing cell counts by 5%, 10%, 20% or 30%, in various embodiments, after a 24-hour incubation in cell culture media at 37° C. In certain embodiments, an inner surface of the wells comprises coating 120 having a metal and a dielectric material. In various embodiments, the described inner surface is present only on the bottom inner surface of the wells, or on both the bottom inner surface and the inner surface of the walls of the wells.

In certain embodiments, utensil 100 may comprise a multi-well plate, for Example one indicated for multiplex ELISA. Multiplex ELISA arrays are quantitative ELISA-based tests where a number of distinct capture antibodies have been absorbed to each well of a 96-well plate in a defined array. These arrays are composed of specific spots with 200-400 μm diameters and a pitch of several hundred μm between spots. Each spot represents a different distinct capture antibody population. Utensil 100 may be arranged to support any number of spots (e.g. 4-spot, 7-spot, 10-spot) and may comprise coating 120 in various patterns for each number of spots.

In certain embodiments, the micro spots have a tiny ridge that borders the binding area (the spot) from the non-binding area. Alternatively or in addition, both the spots and the non-binding area are black (i.e. coated by coating 120). Alternatively or additionally, the surface outside the spots is non-binding for biological molecules and is engineered to minimize back-scattered light and crosstalk between the spots.

In certain embodiments, utensil 100 comprises an embossed foil (FIG. 1D). In certain embodiments, the foil is embossed with holes or wells in a regular pattern suitable for a multi-well plate processing apparatus.

In certain embodiments, utensil 100 comprises a traditionally-shaped multi-well plate (FIG. 1A). Such plates have a frame, typically substantially rectangular or square, which contains a plurality of reagent wells. The frame has a surface, often flat or complementary to a multi-well plate processing apparatus, on which the frame is intended to rest. Thus this surface will be on the bottom when the plate is in its intended orientation. The openings of the wells face upward in this orientation. In some embodiments, the substrate of the plate, often composed of a polymer, imparts its overall shape, and the substrate is coated with a mixture of metal and dielectric material, or in other embodiments separate layers of metal and dielectric material.

Utensil 100 as a multi-well plate may be manufactured by coating a polymer having a desired shape with a coating described herein. The plate may be any of the plates described herein, having any of the embodiments described herein, each of which is considered a separate embodiment. The coating may be any of the coatings described herein, having any of the embodiments described herein, each of which is considered a separate embodiment.

In certain embodiments, utensil 100 comprises a continuous array tape, wherein the surface of the tape is intended for contact with a biological sample and comprises coating 120 comprising metal and dielectric material. In certain embodiments, the tape is a multi-well, continuous-array tape, in certain embodiments a multi-well continuous-array assay tape.

Utensil 100 as a continuous array tape may be manufactured by coating a polymer having a desired shape with a coating described herein. The tape may be any of the tapes described herein, having any of the embodiments described herein, each of which is considered a separate embodiment. The coating may be any of the coatings described herein, having any of the embodiments described herein, each of which is considered a separate embodiment. In certain embodiments, a metal layer is first deposited onto a substrate by means of reactive sputtering in a technique which enhances the porosity and active surface area within the layer. Following the metal deposition, the substrate enters a second process zone (within the same vacuum chamber or as a separate production step), where an additional layer is produced by a reactive evaporation of metal and metal oxide admixtures by a thermal evaporation process. In a non-limiting example, the production method may increase the surface area of a substrate, by: (a) placing the substrate in an inert atmosphere, having a pressure of between 10⁻³ torr and 10⁻² torr, into which oxygen has been introduced at a pressure of from one to two orders of magnitude less than the inert atmosphere pressure; and (b) evaporating valve metal(s) only, onto a heated substrate under the oxygen-containing inert atmosphere, whereby the product comprises a mixture of fractal surface structure including at least one valve metal and at least one valve metal oxide deposited on the substrate.

In certain embodiments, utensil 100 comprises continuous strips, typically composed of a polymer, that are serially embossed with reaction wells. Utensil 100 may comprise, for Example, 96- or 384-well arrays, or have a custom array format, with specified well geometries, well volumes and tape reel lengths. Utensil 100 may comprise a continuous polypropylene strip, serially embossed with reaction wells in customized volumes and formats. Coating 120 may be applied to any part of utensil 100, such as the whole surface, walls, bottoms, sides, tracks or any other geometrical feature of utensil 100 or part thereof.

In certain embodiments, utensil 100 comprises a foil metal insert for a multi-well plate, in some embodiments a multi-well assay plate (FIG. 1A or 1F), wherein the surface of the insert intended for contacting a biological solution comprises coating 120 having a metal and a dielectric material. For example, plates having large wells may be accommodated with coated inserts for reducing background fluorescence.

In certain embodiments, utensil 100 comprises a foil that is used as a backing (FIG. 1F) that is attached to the back (bottom) surface of a multi-well plate, for Example a plate composed of a polymer, in order to reduce fluorescence. In some embodiments, the wells in the frame may be bottomless, with the backing, once attached, providing the bottom surface. In certain embodiments, the backing may be manufactured either together with the frame, or separately from the frame, for subsequent attachment. Once attached, the backing may or in other embodiments may not be removable. In certain embodiments, the entire plate is disposable.

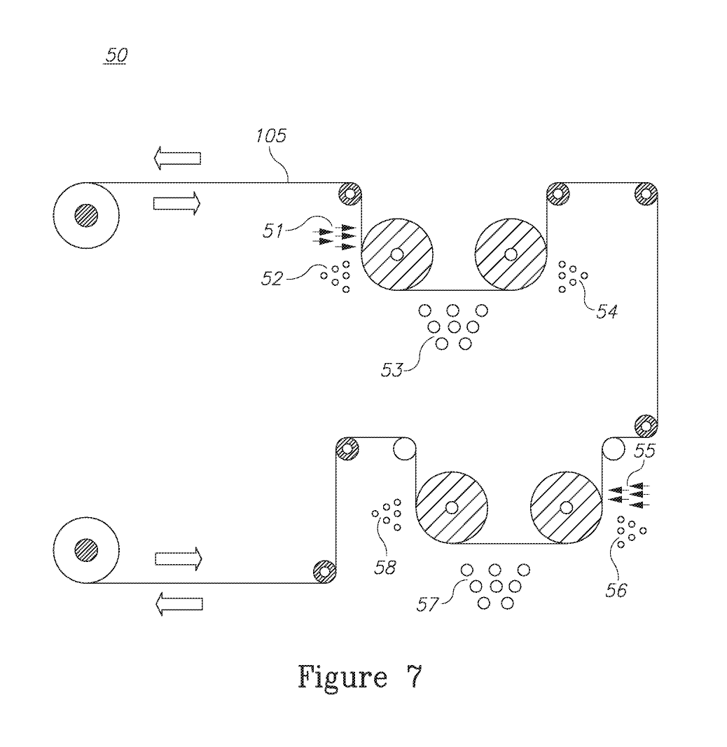

In certain embodiments, layers 122, 124 may be deposited onto a thin foil substrate in a roll-to-roll process (see FIG. 7). Similar process steps may performed in-line inside a unique web coater that is capable of simultaneously performing all process steps onto both sides of the substrate, which is typically Ti or Al foil. The metallic foil substrate may be processed in continuous rolls, which allows significant cost reduction through the high volume production process. FIG. 7 illustrates a scheme of an in-line process carried out inside a vacuum chamber 50, which enables production of coated foil in mass production volumes, according to some embodiments of the invention. Foil 105 may be run between rollers and receive deposited materials and/or reactants according to the following operations: plasma cleaning 51, sputtered layer 52 (optional), addition 53 of porous black layer which may contain e.g. Al+alumina, optional addition 54 of an additional layer (e.g. titanium or a metal oxide, e.g. alumina) In certain embodiments, the same processes may be applied to the other side of foil 105 (operations 55-58 correspond to operations 51-54, respectively). The product of the process, which can be made inter alia as described in Example 1, may be coated on one or both sides and the coating may be responsible for the absorption of stray light and IR radiation. Foil 105 may be applied from continuous roll of substrate material onto which coating 120 is applied. In certain embodiments, the foil may be used on its own (e.g., embossed) or be attached to form bottoms of a multiwall plate.

In certain embodiments, utensil 100 comprises an embossed foil, adapted for use as a multi-well assay plate (FIG. 1D). The foil may be patterned in a pattern suitable for a multi-well plate-reading apparatus, for Example by embossing. In some embodiments, utensil 100 as foil is a double-side-coated foil. In certain embodiments, the foil may be cut into strips/units, which are covered by specific ligands. Utensil 100 as a multi-well plate or an embossed foil may be adapted to perform cell-based assays, which are used, e.g., for drug discovery; drug target identification, screening and validation; studies of biological pathways, analysis of the mode of action of pharmaceutical active agents, and toxicology studies, often with 6-, 12-, 24-, 48-, and 96-well plates, typically using absorbance, fluorescence, or luminescence. Often, luminescence gives the highest sensitivity and the greatest dynamic range.

In certain embodiments, utensil 100 comprises a foil in roll form suitable for use as a multi-well plate (if cut into segments) or multi-well tape, which includes a single porous, essentially continuous, vacuum-deposited ceramic layer 122, disposed on at least one side of, and supported by, a porous or non-porous substrate, ceramic layer 122 may comprise at least one metal oxide selected from the group consisting of aluminum, titanium, tantalum, niobium, zirconium, silicon, thorium, cadmium and tungsten oxides, wherein, optionally, at least one of the following conditions is also fulfilled, namely: (a) ceramic layer 122 may comprise at least one rare earth metal; (b) ceramic layer 122 may have a fractal surface structure; and/or (c) the non-porous substrate is selected from aluminum and polymeric substrates.

In some embodiments, the described foil is coated with spots of detection molecules, for Example DNA, peptides, monoclonal antibodies (mAbs), or recombinant proteins, using any of the technologies described or referenced herein. Usage is, in some embodiments, in combination with a microwell plate, wherein the bottom of the plate consists of the patterned foil that is attached to the bottomless frame. In addition to more conventional methods, readouts from such plates may comprise a method selected from imaging, SEM, and atomic force microscopy, each of which is considered a separate embodiment.

In certain embodiments, utensil 100 as foil may be used for coating glass slides or microarrays. In some embodiments, spots with a specific surface morphology, such as particular structures or micropores, may create a highly adsorptive porous surface to improve protein loading, DNA binding, or cell attachment. In some embodiments, modified surfaces with high affinity for polar groups may be used for adsorption of antibodies and glycoproteins, while surfaces modified with negatively-charged groups or coated with an extracellular matrix are used for cell attachment. In some embodiments, creation of hydrophobic and hydrophilic areas or spots on the foil enables the precise, accurate and reproducible positioning of different sample types. In some embodiments, utensil 100 as the described foil is flexible. In certain embodiments, utensil 100 may comprise the microarray as a non-contact micro-dispensing system designed specifically for dispensing sub-nanoliter to nanoliter volumes to create dense arrays. In certain embodiments, the described microarray is particularly adapted for covalent binding of RNA, DNA, antibodies, peptides, glycoproteins, or glycans. In some embodiments, porous surfaces are particularly suitable for binding antibodies. In certain embodiments, utensil 100 may be used for producing microarrays used for molecular diagnosis, e.g., in allergology or in any other field such as screening, personalized medicine, diagnostics and therapeutic decision making and monitoring. Patterning coating 120 on microarrays may also be advantageous in a biological samples printing process, by directing the printed drops into their destined location using hydrophobic and/or hydrophilic features of coating 120 or coating regions.

In certain embodiments, utensil 100 comprises slides which are suitable for an application selected from pathology, cytology, clinical microbiology, and immunohistochemistry (FIGS. 1B, 1C). In certain embodiments, utensil 100 comprises glass slides which are printed with ink. In certain embodiments, the glass slides are printed with ring marks or wells that are formed with highly water-repellent fluororesin-based ink suitable for fluorescence antibody methods and in situ hybridization. The glass slides may have any patterns, e.g., flow channels (illustrated schematically in FIG. 1C) having any degree of complexity. The high printing and ablation resolutions enabled by coating 120 allow producing very fine details and large complex structures on a small area.

In certain embodiments, utensil 100 comprises a lab-on-a-foil, wherein the surface of the lab-on-a-foil intended for contacting a biological solution comprises coating 120 having a metal and a dielectric material. The described labs-on-a-foil may be used, in certain embodiments, for various assays, including PCR and immunoassays, and in other embodiments may be treated with embossing at predefined positions, for precise, accurate and reproducible positioning of the samples. In some embodiments, the labs-on-a-foil are flexible.

In certain embodiments, utensil 100 comprises a membrane adapted for separation of biological materials, composed of a ceramic or in another embodiment etched-foil substrate, wherein the substrate is mesh or in another embodiment porous, wherein the surface on at least one side of the membrane is porous coating 120 that comprises a metal and a dielectric material.

In certain embodiments, the substrate is alumina. In certain embodiments, the surface is a black surface. Alternatively or in addition, both sides of the membrane may be coated with coating 120. In certain embodiments, the entire thickness of the membrane is porous, to enable penetration of aqueous solution thereinto. In certain embodiments, utensil 100 as membrane may in some embodiments have various pore sizes: Some standard pore sizes are 0.02 μm, 0.1 μm, and 0.2 μm.

In some embodiments, such membranes are suited for a wide range of laboratory filtration applications, for Example: HPLC mobile phase filtration and degassing; Ultra cleaning of solvents; Gravimetric analysis; Liposome extrusion; Scanning electron microscopy studies; Bacterial analysis by epifluorescence light microscopy; Micrometer and nanometer filtration; Metal nanorods formation.

In certain embodiments, utensil 100 as membrane may comprise a multi-layer system of at least two porous, essentially continuous, vacuum-deposited ceramic layers 122A, 122B (FIG. 2F) that are disposed on at least one side of, and supported by, porous substrate 102. Ceramic layers 122 may comprise at least one metal oxide selected from the group consisting of aluminum, titanium, tantalum, niobium, zirconium, silicon, thorium, cadmium and tungsten oxides, wherein between any successive ceramic layers, there is disposed a vacuum-deposited metallic layer 124, wherein the porosity and/or average pore width of the metallic layer is less than the porosity and/or average pore width of the ceramic layer, at least one of the following conditions also optionally being fulfilled: (a) at least one ceramic layer 122 has a fractal surface structure; (b) ceramic layer 122 consists essentially of a mixture of metal(s) and oxide(s) thereof; (c) the membrane has sufficient flexibility enabling it to be rolled up and unrolled; (d) there is disposed on the surface of any outermost ceramic layer a vacuum-deposited metallic layer, wherein the porosity and/or average pore width of the metallic layer is less than the porosity and/or average pore width of the outermost ceramic layer.

Without being bound by theory, in utensil 100 configured as such membrane, substrate 102 may operate as a mechanical support for the membrane which allows free flow of fluid therethrough. The substrate generally defines obverse and reverse sides and the supported ceramic layer is disposed either on one side only, or on both sides, of the substrate. Where the supported ceramic layer is disposed on one side only of the substrate, a vacuum-deposited metallic layer may be disposed on the surface of the ceramic layer. Where the supported ceramic layer is disposed on both sides of the substrate, thus defining two ceramic layer surfaces, a vacuum-deposited metallic layer or layers may be disposed on at least one of the two surfaces.

In some embodiments of the membrane, at least one of the following further conditions is fulfilled: (i) the vacuum-deposited ceramic layer has been deposited by physical vapor deposition; (ii) the substrate is a metallic substrate; (iii) the ceramic layer has a fractal surface structure selected from dendrite, cauliflower-like and coral-like fractal surface structures; (iv) the ceramic layer consists essentially of a mixture of aluminum metal and alumina; (v) the supported ceramic layer is disposed on both sides of the substrate, both ceramic sides being also bonded to each other through the pores of the substrate, thereby imparting improved mechanical strength to the membrane.

In some embodiments, the raw materials and the process conditions are selected so as to impart a fractal surface structure to ceramic layer 122, that is, a surface composed of fractals.

In certain embodiments, utensil 100 may comprise a biochip, for Example a DNA microarray, protein microarray, or lab-on-a-chip, wherein the surface of the biochip intended for contacting a biological solution comprises a metal and a dielectric material. Microarrays involve attaching biological materials to a planar surface in an ordered manner Utensil 100 may thus be used for sample preparation, sample analysis, and diagnostics.

In certain embodiments, utensil 100 may be configured as a lab-on-a-chip with respect to any of the following technologies, as non-limiting Examples: PCR analysis (Amplification of a single or a few copies of a piece of DNA across several orders of magnitude, generating thousands to millions of copies of a particular DNA sequence), Electrophoresis (Dispersed particles e.g. DNA, RNA, Proteins, relative to a fluid under the influence of a spatially uniform electric field), Gene ID (Microarray products to check IDs of organisms in food and feed (like GMO), mycoplasms in cell culture, or pathogens for disease detection, mostly combining PCR and microarray technology), Comparative genomic hybridisation (Assessing genome content in different cells or closely related organisms), Chromatin immuno-precipitation on chip (DNA sequences bound to a particular protein can be isolated by immunoprecipitating that protein (ChIP), these fragments can be then hybridized to a microarray (such as a tiling array) allowing the determination of protein binding site occupancy throughout the genome. Example protein to immunoprecipitate are histone modifications (H3K27me3, H3K4me2, H3K9me3, etc.), Polycomb-group protein (PRC2:Suz12, PRC1:YY1) and trithorax-group protein (Ash1) to study the epigenetic landscape or RNA Polymerase II to study the transcription landscape), and Immunoassays (Binding between an antigen and its homologous antibody in order to identify and quantify the specific antigen or antibody in a sample).

In some embodiments, utensil 100 may be configured as a protein microarray apparatus having coating 120 configured to enable proteins to maintain their secondary and tertiary structure and thus their biological activity and their interactions with other molecules. In certain embodiments, the shelf-life of the product, without the proteins denaturing, is enhanced. In certain embodiments, the biological molecules are readily extracted from the foil surface (coating 120).

In certain embodiments, utensil 100 may be configured as a protein microarray with respect to any of the following technologies, as non-limiting Examples: Analytical array/capture array (In this technique, a library of antibodies, aptamers or affibodies is arrayed on the support surface. These are used as capture molecules since each binds specifically to a particular protein. The array is probed with a complex protein solution such as a cell lysate. Analysis of the resulting binding reactions using various detection systems can provide information about expression levels of particular proteins in the sample as well as measurements of binding affinities and specificities. This type of microarray is especially useful in comparing protein expression in different solutions. For instance the response of the cells to a particular factor can be identified by comparing the lysates of cells treated with specific substances or grown under certain conditions with the lysates of control cells. Another application is in the identification and profiling of diseased tissues), Functional protein microarray/target protein array (These are constructed by immobilizing large numbers of purified proteins and are used to identify protein-protein, protein-DNA, protein-RNA, protein-phospholipid, and protein-small molecule interactions, to assay enzymatic activity and to detect antibodies and demonstrate their specificity. They differ from analytical arrays in that functional protein arrays are composed of arrays containing full-length functional proteins or protein domains. These protein chips are used to study the biochemical activities of the entire proteome in a single experiment) and Reverse phase protein array (These arrays involve complex samples, such as tissue lysates. Cells are isolated from various tissues of interest and are lysed. The lysate is arrayed onto the microarray and probed with antibodies, often dye-labeled, against the target protein of interest. These antibodies are typically detected with chemiluminescent, fluorescent or colourimetric assays. Reference peptides are printed on the slides to allow for protein quantification of the sample lysates. RPAs allow for the determination of the presence of altered proteins or other agents that may be the result of disease. Specifically, post-translational modifications, which are typically altered as a result of disease can be detected using RPAs).

In some embodiments, utensil 100 may be configured as a DNA microarray apparatus having coating 120 configured to enable DNA molecules to maintain their structure and thus their biological activity and their interactions with other molecules. In certain embodiments, the shelf-life of the product, without the DNA molecules denaturing, is enhanced. In certain embodiments, the biological molecules are readily extracted from the foil surface (coating 120). In certain embodiments, utensil 100 may be configured as a RNA microarray apparatus.

In certain embodiments, utensil 100 may be configured as a DNA microarray with respect to any of the following technologies, as non-limiting Examples: SNP detection (Identifying single nucleotide polymorphism among alleles within or between populations. Several applications of microarrays make use of SNP detection, including genotyping, forensic analysis, measuring predisposition to disease, identifying drug-candidates, evaluating germline mutations in individuals or somatic mutations in cancers, assessing loss of heterozygosity, or genetic linkage analysis), Gene expression profiling (In an mRNA or gene expression profiling experiment the expression levels of thousands of genes are simultaneously monitored to study the effects of certain treatments, diseases, and development stages on gene expression. For Example, microarray-based gene expression profiling can be used to identify genes whose expression is changed in response to pathogens, cancer or other organisms by comparing gene expression in infected/diseased to that in uninfected/healthy cells or tissues), Alternative splicing detection (An exon junction array design uses probes specific to the expected or potential splice sites of predicted exons for a gene. It is of intermediate density, or coverage, to a typical gene expression array (with 1-3 probes per gene) and a genomic tiling array (with hundreds or thousands of probes per gene). It is used to assay the expression of alternative splice forms of a gene. Exon arrays have a different design, employing probes designed to detect each individual exon for known or predicted genes, and can be used for detecting different splicing isoforms), and a Fusion gene microarray (A fusion gene microarray can detect fusion transcripts, e.g. from cancer specimens.

The principle behind this is building on the alternative splicing microarrays. The oligo design strategy enables combined measurements of chimeric transcript junctions with exon-wise measurements of individual fusion partners).

In certain embodiments, utensil 100 comprises a microscope pad, wherein the surface of the pad comprises coating 120 of a metal and a dielectric material, as described herein.

In certain embodiments, utensil 100 comprises a microbead, wherein the surface of the microbeads comprises coating 120 with a metal and a dielectric material, as described herein. Utensil 100 as microbeads may be used for multiplex assays. In certain embodiments, the coated beads described herein are manufactured similarly to the coated foil described herein, except that small fragments of about 5 micron are used.

In certain embodiments, utensil 100 comprises other life science products, namely biochips, sheets, and slides, wherein the surface of the products comprises a metal and a dielectric material, as described herein.

Any of the embodiments of utensil 100 described above, such as multi-well plate, foil, embossed foil, continuous array tape, foil metal insert, lab-on-a-foil, DNA or protein microarrays, slide, glass slide, flow cell, microscope pad, microbead, membrane, biochip and life science products, may exhibit any of the arrangements of coating 120 described e.g., below, namely the arrangement of the metal and the dielectric material; arrangements of the substrate (if a separate substrate is present) and the coating; methods of coating the substrate, if present; types of metals and dielectric materials; levels of fluorescence, reflectance, cytotoxicity, and damage threshold; porosity size and pattern; conjugation to other molecules; surface area; and combinations thereof, each being considered as being a separate embodiment.

Table 1 reviews non-limiting examples for biological molecules that may be bonded or linked to coating 120, in the fields of immunology, molecular biology and cell culture, according to some embodiments of the invention.

TABLE 1
Overview of characteristic applications and coating specification in terms of
surface characteristics or bonded biological molecules, in the fields of
immunology, molecular biology and cell culture, according to some
embodiments of the invention.

| Characteristics | Specialized surface/functional groups |
|---|---|
| Covalent coupling of peptides, | Secondary amine surface encompass high binding |
| proteins, carbohydrates, and | strength, binding of distinct functional groups, |
| nucleic acids to polystyrene | specific orientation of molecules and improved |
| | stability |
| Adsorption of glycoproteins such | Modified surfaces e.g. on polystyrene base, with a |
| as antibodies to the plastic surface | high affinity for polar groups with |
| excellent -> for double antibody | sensitivity |
| “sandwich” immuno assays e.g. | |
| ELISA | |
| Binding of molecules of an | Lower affinity for proteins e.g. polystyrene surface |
| intermediate | treated |
| hydrophobic/hydrophilic nature -> | |
| for assays using human sera, as | |
| the signal to noise ratio tends to | |
| be improved | |
| Avoiding of non-specific binding | Very low affinity for proteins or DNA e.g. with |
| of DNA or proteins -> ideal for | surfaces from a specially formulated polyethylene |
| solution based and DNA probe | resin |
| assays | |
| No affinity/adhesion for/of | Very hydrophilic polystyrene surface |
| hydrophobic analytes -> for the | |
| immobilisation of highly polar | |
| molecules or for homogenous | |
| assays | |

Medium affinity to polar groups -> for the use with more hydrophobic molecules,
such as viral antigens and in double antibody “sandwich” assays.

| | |
|---|---|
| Binding and amplification of DNA | Covalent binding of DNA e.g. by carbodiimide |
| | mediated condensation; thermally stable surface |
| Growth surface area, which has | Negatively charged surface that is suitable for cell |
| been modified, to a hydrophilic | attachment and growth. Generated by alteration of |
| state for adherent cells. Other | the plastic surface by either a corona or plasma |
| characteristics are greater oxygen | discharge treatment. Both methodologies add |
| permeability, and good chemical | oxygen in the form of carboxyl groups into the |
| compatibility. | molecular structure of the plastic surface. |
| | For optimum attachment of cells, plates are often |
| | coated with extra cellular matrix (ECM) protein |
| | such as collagen, laminin, fibronectin, Poly-D |
| | Lysine etc. |
| No cell attachment to a growth | Untreated surface |
| substrate to ensure proliferation | |
| for suspensions cells | |
| Surface modification to achieve a | Specific polyester film for cell culture cover slips |
| hydrophilic state for cell | |
| adherence and growth. Highly | |
| resistant surface to solvents. -> for | |
| specific applications like electron | |
| microscopy | |

Table 2 reviews non-limiting examples for biological molecules that may be bonded or linked to coating 120, according to some embodiments of the invention. Table 2 relates characteristics of coating 120, in particular of porous ceramic layer 122, to classes of biological molecules which may be bonded upon the surface porous ceramic layer 122. The inventors have discovered that the surface of coating 120, in particular of porous ceramic layer 122, may be adapted to these classes of biological molecules and hence to enable the binding thereof to coating 120 and to utensil 100 (see details nd examples below). The performance criteria in Table 2 were used to quantify the binding efficiency of the biological molecules to various samples.

TABLE 2
Exemplary biological molecules that may be bonded or linked to the coating, and related
coating characteristics, according to some embodiments of the invention.

| | | | | | Standard |
|---|---|---|---|---|---|
| Sample Molecule | | | | | performance |
| class | Application | Surface | Characteristics | Binding interaction | criteria |
| Proteins and large | General | Medium | Hydrophobic | Binds biomolecules | Standard |
| | assays | | | through | binding |
| molecules (>20 kD) | Immunoassays | binding | | passive interactions | capacity: |
| | | | | | 100-200 ng |
| e.g. Antibodies | (EUSA/RIA) | | | | IgG/cm² |
| Medium (>lOkD) | Immunoassays | High | Hydrophobic | Different binding | Standard |
| and large | | binding | and | surfaces, e.g. | binding |
| Proteins that possess | (ELISA/RIA) | | ionic | covalently-linked | capacity: |
| ionic | | | | maleimide | 400-500 ng |
| groups and/or +/− | | | | groups that covalently | IgG/cm² |
| hydrophobic regions | | | | couple to sulfhydryl | |
| (positively charged) | | | | groups via SH | |
| e.g. antibodies, | | | | moieties (->Abs), or | |
| immuno globulines, | | | | hydrazide groups | |
| albumines, matrix | | | | covalently coupled to | |
| proteins | | | | carbohydrate goups | |
| | | | | (->carbohydrates, | |
| | | | | glycoproteins) | |
| Nucleic acids, | Homogeneous | Non- | Nonionic | minimises molecular | At least 90-95% |
| | assays | binding | | | |
| Proteins | e.g. | | hydrophilic | interactions; Ideal for | reduction of |
| | fluorescent | | | reducing | non- |
| | assays | | | protein and nucleic | specific |
| | | | | acid binding | binding of |
| | | | | at low concentrations, | protein |
| | | | | and | compared to |
| | | | | increasing/enhancing | untreated |
| | | | | assay | standard |
| | | | | signal to noise | polystyrene |
| Adherent cell culture | Cell and | Binding | Hydrophilic | Negative charged | |
| | tissue | | | (carboxyl | |
| | culture | | | groups) for cell | |
| | | | | attachment | |
| Suspended cell | Cell and | Non- | Untreated | | |
| culture | tissue | binding | | | |
| | culture | | | | |
| Cell culture | Cell-based | Binding | Hydrophilic | Uniform a net positive | |
| | assays | | and | charge | |
| | | | ionic | Enhancing cell | |
| | | | | attachment, | |
| | | | | growth and | |
| | | | | differentiation | |

### Coating Configurations and Production

In certain embodiments, coating 120 may comprise a mixture of a metal and a dielectric material. In some embodiments, the surface of biological apparatus 100 that contacts the biological sample (e.g., the inner surface of utensil 100, in the case of a multi-well plate) may be composed entirely of metal and dielectric, while in certain embodiments, other components may be present. In certain, more specific embodiments, the ratio of the dielectric material to the metal is between 80:20 and 90:10.

Utensil 100 may either be made, at least partially, of the mixture of metal and dielectric material, or may be coated with the mixture (utensil 100 may then be made of a polymer or a metal as non-limiting Examples. Exemplary, non-limiting embodiments of polymers are plastics, for Example polypropylene, polystyrene, polycarbonate, and flexible polyvinyl chloride (vinyl).

In certain embodiments, the metal and the dielectric material may be present as separate layers, one of which forms the immediate surface, and the other of which is directly under it (as illustrated e.g. in FIG. 2G by layers 124 and 122, respectively). In certain embodiments, the metal layer may be a core of coating 120 and be covered by the dielectric (e.g., oxide) layer. Such embodiments are not precluded by reference to the surface as “comprising a metal and a dielectric material” or similar language, since “surface” in the context of these embodiments refers to the immediate surface and a layer directly under it, up to a thickness of 250 microns. In some embodiments, there is an intervening layer between the layers of the metal and the dielectric material, while in certain embodiments, the layers of metal and dielectric material are in direct contact with each other. Typically, in such arrangements, the dielectric material is on the surface and thus is in position to contact the biological sample.

In some embodiments, utensil 100 comprises separate layers of metal and dielectric material 124, 122 respectively. In certain embodiments, utensil 100 comprises layer of metal 124, layer of dielectric material 122, and an intervening layer. In certain embodiments, the plate comprises a substrate that is coated with the layers. In certain embodiments, the plate comprises a substrate that is coated with the layer of metal, the layer of dielectric material, and an intervening layer.

In certain embodiments utensil 100 comprises a substrate, often consisting of a polymer, that is coated with layer 120 as a mixture of metal and dielectric material. Various methods may be used to coat or cover the substrate. In some embodiments, the mixture is added by vacuum deposition, as defined herein, or in certain embodiments, by other methods described herein. In various embodiments, the substrate may or may not be patterned prior to application of the coating. In some embodiments, coating 120 is patterned after its application to the substrate (e.g. by laser ablation).

Ceramic layer 122 may comprise a dielectric material such as a metal oxide. In certain, more specific embodiments, the metal oxide may be an oxide of the same metal that is present in metal layer 124 of coating 120. In certain embodiments, the oxide is an oxide of another metal. In certain embodiments, metal layer 124 may be selected from the group consisting of aluminum and titanium. In more specific embodiments, the metal is selected from the group consisting of aluminum and titanium, and the metal oxide is an oxide of aluminum or titanium. In even more specific embodiments, the metal is aluminum and the dielectric material is an aluminum oxide. Alternatively, the metal is titanium and the dielectric material is a titanium oxide. In certain embodiments, the coating consists of an aluminum/alumina mixture. In certain embodiments, the alumina is gamma (γ) alumina or alpha (α) alumina.

In certain embodiments, metal layer 124 may comprise a valve metal such as aluminum, titanium, tantalum, niobium, zirconium, silicon, thorium, cadmium and tungsten. In certain embodiments, the described coating consists of a valve metal and an oxide thereof, the valve metal being selected from aluminum, titanium, tantalum, niobium, zirconium, silicon, thorium, cadmium and tungsten.

In certain embodiments, utensil 100 may be coated with a multi-layer coating 120, in which ceramic later 122 comprises at least one inner layer 122B of vacuum-deposited oxide of at least one metal and at least one outer layer 122A of a vacuum-deposited mixture of at least one valve metal with at least one valve metal oxide (FIG. 2F). In certain embodiments, the vacuum-deposited mixture may be formed by reactive vapor deposition of at least one valve metal in presence of oxygen under predetermined conditions adapted for the formation of the mixture. Alternatively or in addition, the vacuum-deposited mixture is formed by evaporating at least one valve metal in an inert atmosphere at a pressure of between 10⁻³ torr and 10⁻² torr, into which oxygen has been introduced at a pressure of from one to two orders of magnitude less than the inert atmosphere pressure. In more specific embodiments, coating 120 may be further characterized by any of the following features: (a) the at least one valve metal is selected from aluminum and titanium; and (b) the vacuum-deposited mixture is optically black. In certain embodiments, the light absorbing layer exhibits a deep black color. Alternatively, its optical characteristics can be tailored to the desired application.

In certain embodiments, the ceramic and the metallic layers (122, 124 respectively) may be fabricated by a vacuum deposition technique, for Example physical vapor deposition (PVD) such as thermal evaporation, electron-beam evaporation, or sputtering, or chemical vapor deposition. Both layer types (ceramic and metallic) can be produced by the same deposition technique or in certain embodiments by different deposition techniques.

In some embodiments, coating 120 may comprise porous, essentially continuous, vacuum-deposited ceramic layer 122, supported by a substrate. Ceramic layer 122 may at least one metal oxide selected from the group consisting of oxides of aluminum, titanium, tantalum, niobium, zirconium, silicon, thorium, cadmium and tungsten, wherein at least one of the following conditions is fulfilled: (a) the ceramic layer has a fractal surface structure; (b) the ceramic layer consists essentially of a mixture of metal(s) and oxide(s) thereof; (c) the substrate has sufficient flexibility enabling it to be rolled up and unrolled; and/or (d) there is disposed on the surface of the ceramic layer a vacuum-deposited metallic layer, wherein the porosity and/or average pore width of the metallic layer is less than the porosity and average pore width of the ceramic layer.

In certain embodiments, ceramic layer 122 used comprises at least one metal oxide selected from the group consisting of oxides of aluminum, titanium, tantalum, niobium, zirconium, silicon, thorium, cadmium and tungsten and is produced by a vapor deposition technique. In some embodiments, metal oxide(s) of the ceramic layer is/are produced as a result of a chemical reaction occurring in the vacuum environment between a metal which is converted from the solid phase to the vapor phase, e.g. by thermal evaporation, and an oxidizing agent, e.g. gaseous mixture comprising oxygen. Thus the process that referred to as “deposition” comprises the following stages: changing the state of the metal from the solid phase to the gaseous phase, chemical reaction, and the deposition stage proper. The process conditions can be chosen so as to produce a coating composed of metal oxide(s) only, or a coating composed of a mixture of metal oxide(s) with metal(s). Thus, in addition to metal oxides the ceramic layer can contain metals, such as aluminum, titanium, tantalum, niobium, zirconium, silicon, thorium, cadmium and tungsten. Metal component(s) of the ceramic coating may improve its mechanical strength, and as a result the mechanical strength of the entire device. The preferable material of the ceramic layer in this case is aluminum oxide (alumina) or a mixture of alumina with aluminum.

Coating 120 may be multilayered and double sided, as illustrated in FIG. 2E (one foil 102 with ceramic layers 122A, 122B on both of its sides). In certain embodiments, there is disposed on the surface of the ceramic layer a vacuum-deposited metallic layer, wherein the porosity and/or average pore width of the metallic layer is less than the porosity and/or average pore width of the ceramic layer. Where the supported ceramic layer is disposed on both sides of the substrate, for Example in the case of an embossed foil used as a multi-well plate, there are two ceramic layer surfaces. In such embodiments, there may be disposed on at least one of the two surfaces a vacuum-deposited metallic layer or layers, wherein the porosity and/or average pore width of the metallic layer(s) is less than the porosity and average pore width of the ceramic layer.

In certain embodiments of the above-described coatings 120, at least one of the following further conditions may be fulfilled: (i) vacuum-deposited ceramic layer 122 may be deposited by physical vapor deposition (PVD); (ii) substrate 102 may be a polymeric substrate; (iii) ceramic layer 122 may have a fractal surface structure selected from dendrite, cauliflower-like and coral-like fractal surface structures; and/or (iv) ceramic layer 122 consists essentially of a mixture of aluminum metal and alumina.

In some embodiments, the raw materials and the process conditions are selected so as to impart a fractal surface structure to ceramic layer 122, that is, a surface composed of fractals. Fractals are mathematical objects that exhibit self-similarity, so that the parts are self-similar to the whole. This self-similarity feature implies that fractals are essentially scale-invariant—one cannot in principle distinguish a small part from the larger structure, e.g. a tree branching process. Coatings with a fractal surface structure are characterized, in some embodiments, by pores of diverse width in the sense that the width of the pore canal covers a relatively wide range of values. Fractal-structured coatings can be better understood from the following explanation, in which a certain type of fractal structure, namely the cauliflower-like structure, is taken as an Example. Substrates with ceramic layer 122 having a cauliflower-like structure are schematically illustrated in FIG. 2A-2G. Ceramic layer 122 is characterized by hemispherical bodies of different sizes attached to each other in decreasing size order, thereby increasing the surface area and porosity of later 122. The structure of layer 122 may be described as a plurality of cauliflower “bodies” 6 bordering each other at pores 123B. Each “body” consists of a “head” and “florets”, the “florets” are branched off the “head” to which they are attached. Each “floret” in its turn has smaller “florets” etc. Thus, the structure is characterized in that each child floret is self-similar to its parent (i.e., the floret to which it is attached). Each cauliflower “head” can be considered as a grandparent with respect to the floret of any level (generation). The described structure resembles a tree structure in which the trunk of the tree corresponds to the head of the cauliflower, and the branches of the tree correspond to the florets. Other fractal structures of the ceramic vacuum deposited coatings may be of dendrite (FIG. 3A) and coral-like (FIG. 3B) types. It is noted that the geometric shape of fractals (i.e., fractal “bodies”) for dendrite and coral-like types are different from each other and from the cauliflower-like type, but all three types of structures are characterized by self-similarity.

In certain embodiments, ceramic layer 122 may have a nano-structure with porosity in the 20-100 nm range and have various micro-structures, density factors and stoichiometries. In non-limiting Examples, the thickness of ceramic layer 122 may range between 30 and 225 μm, the density may range between 1.8-2 g/cm³, ceramic layer 122 may be produced upon metal layer 124 ranging in thickness between 32 and 120 μm. In non-limiting Examples, ceramic layer 122 may be made of Al₂O₃ or of hydrated aluminum (Al₂Oₓ, where the stoichiometry of oxygen to aluminum has not been precisely determined).

In certain embodiments, coating 120 may be suitable for biological apparatuses. In various embodiments, the coatings can be manufactured as sterile/clean room class I, can be heat-sterilized, can withstand temperatures of 4° K-400° C., are readily cleaned and recycled, and readily printable and patternable (for Example by laser ablation to remove the coating, and by photolithography/etching, etc.), and/or are suitable for lab-on-a-foil/chip applications.

In certain embodiments, coated utensil 100 exhibits very low fluorescence. In more specific embodiments, utensil 100 may exhibit very low induced fluorescence over a defined range of irradiation conditions. For Example, in one embodiment utensil 100 has a fluorescence rate of 10⁻⁹ or less of the number of incoming photons, over a range of incoming wavelengths of 200-280 nm and a range of fluorescence wavelengths of 280-380 nm. In certain embodiments, utensil 100 has a fluorescence rate of 10⁻⁸ or less of the number of incoming photons, under the same condition. In certain embodiments, the fluorescence rate is 7×10⁻⁹ or less, 5×10⁻⁹ or less, 3×10⁻⁹ or less, 2×10⁻⁹ or less, 1.5×10⁻⁹ or less, 8×10⁴⁰ or less, 5×10⁻⁹ or less, 3×10⁻⁹ or less, 2×10⁻⁹ or less, 1.5×10⁻⁹ or less, or 1×10⁻⁹ or less.

In certain embodiments, coatings 120 are black coatings. Blackness, as used herein, refers to the ability of a surface to transmit or absorb a large fraction of incident radiation (thus reflecting a correspondingly small fraction of the radiation) over a particular range of wavelengths and at a particular angle. In certain embodiments, the surfaces are black with regard to the spectrum of visible light, between 380-740 nanometers (nm). In certain embodiments, the surfaces are black with regard to the entire spectrum of ultraviolet light, from 10-380 nm. In certain embodiments, the surfaces are black with regard to ordinary ultraviolet light, between 120-380 nm. In certain embodiments, the surfaces are black with regard to extreme ultraviolet light, between 10-120 nm. In certain embodiments, the surfaces are black with regard to both visible and ultraviolet light.

In various embodiments, the described coatings reflect less than 1×10⁴, 5×10⁻⁵, 3×10⁻⁵, 2×10⁻⁵, 1×10⁻⁵, 5×10⁻⁶, 3×10⁻⁶, 2×10⁻⁶, 1×10⁻⁶, 5×10⁻⁷, 3×10⁻⁷, 2×10⁻⁷, 1×10⁻⁷, 5×10⁻⁸, 3×10⁻⁸, 2×10⁻⁸, 1×10⁻⁸, 5×10⁻⁹, 3×10⁻⁹, 2×10⁻⁹, 1×10⁻⁹, 5×10⁻¹⁰, 3×10⁻¹⁰, 2×10⁻¹⁰, 1×10⁻⁴, 5×10⁻¹¹, 3×10⁻¹¹, 2×10⁻¹¹, or 1×10⁻¹¹ of the spectrum of visible light at a 90° angle. In certain embodiments, the described coatings reflect less than 1×10⁻⁴, 5×10⁻⁵, 3×10⁻⁵, 2×10⁻⁵, 1×10⁻⁵, 5×10⁻⁶, 3×10⁻⁶, 2×10⁻⁶, 1×10⁻⁶, 5×10⁻⁷, 3×10⁻⁷, 2×10⁻⁷, 1×10⁻⁷, 5×10⁻⁸, 3×10⁻⁸, 2×10⁻⁸, 1×10⁻⁸, 5×10⁻⁹, 3×10⁻⁹, 2×10⁻⁹, 1×10⁻⁹, 5×10⁻¹⁰, 3×10⁻¹⁰, 2×10⁻¹⁰, 1×5×10⁻¹¹, 3×10⁻¹¹, 2×10⁻¹¹, or 1×10⁻¹¹ of the spectrum of ordinary ultraviolet light at a 90° angle. In still embodiments, the described coatings reflect less than 1×10⁻⁴, 5×10⁻⁵, 3×10⁻⁵, 2×10⁻⁵, 1×10⁻⁵, 5×10⁻⁶, 3×10⁻⁶, 2×10⁻⁶, 1×10⁻⁶, 5×10⁻⁷, 3×10⁻⁷, 2×10⁻⁷, 1×10⁻⁷, 5×10⁻⁸, 3×10⁻⁸, 2×10⁻⁸, 1×10⁻⁸, 5×10⁻⁹, 3×10⁻⁹, 2×10⁻⁹, 1×10⁻⁹, 5×10⁻¹⁰, 3×10⁻¹⁰, 2×10⁻¹⁰, 1×10⁻¹⁰, 5×10⁻¹¹, 3×10⁻¹¹, 2×10⁻¹¹, or 1×10⁻¹¹ of the spectrum of extreme ultraviolet light at a 90° angle. In yet embodiments, the described coatings reflect less than 1×10⁻⁴, 5×10⁻⁵, 3×10⁻⁵, 2×10⁻⁵, 1×10⁻⁵, 5×10⁻⁶, 3×10⁻⁶, 2×10⁻⁶, 1×10⁻⁶, 5×10⁻⁷, 3×10⁻⁷, 2×10⁻⁷, 1×10⁻⁷, 5×10⁻⁸, 3×10⁻⁸, 2×10⁻⁸, 1×10⁻⁸, 5×10⁻⁹, 3×10⁻⁹, 2×10⁻⁹, 1×10⁻⁹, 5×10⁻¹⁰, 3×10⁻¹⁰, 2×10⁻¹⁰, 1×10⁻¹¹, 5×10⁻¹¹, 3×10⁻¹¹, 2×10⁻¹¹, or 1×10⁻¹¹ of the spectrum of visible light at a 45° angle. In certain embodiments, the described coatings reflect less than 1×10⁻⁴, 5×10⁻⁵, 3×10⁻⁵, 2×10⁻⁵, 1×10⁻⁵, 5×10⁻⁶, 3×10⁻⁶, 2×10⁻⁶, 1×10⁻⁶, 5×10⁻⁷, 3×10⁻⁷, 2×10⁻⁷, 1×10⁻⁷, 5×10⁻⁸, 3×10⁻⁸, 2×10⁻⁸, 1×10⁻⁸, 5×10⁻⁹, 3×10⁻⁹, 2×10⁻⁹, 1×10⁻⁹, 5×10⁻¹⁰, 3×10⁻¹⁰, 2×10⁻¹⁰, 1×10⁻⁴, 5×10⁻¹¹, 3×10⁻¹¹, 2×10⁻¹¹, or 1×10⁻¹¹ of the spectrum of ordinary ultraviolet light at a 45° angle. In still embodiments, the described coatings reflect less than 1×10⁻⁴, 5×10⁻⁵, 3×10⁻⁵, 2×10⁻⁵, 1×10⁻⁵, 5×10⁻⁶, 3×10⁻⁶, 2×10⁻⁶, 1×10⁻⁶, 5×10⁻⁷, 3×10⁻⁷, 2×10⁻⁷, 1×10⁻⁷, 5×10⁻⁸, 3×10⁻⁸, 2×10⁻⁸, 1×10⁻⁸, 5×10⁻⁹, 3×10⁻⁹, 2×10⁻⁹, 1×10⁻⁹, 5×10⁻¹⁰, 3×10⁻¹⁰, 2×10⁻¹⁰, 1×10⁻⁴, 5×10⁻¹¹, 3×10⁻¹¹, 2×10⁻¹¹, or 1×10⁻¹¹ of the spectrum of extreme ultraviolet light at a 45° angle.

Alternatively or in addition, coating 120 on utensil 100 exhibits hemispherical reflectance of less than 2% over a range of wavelengths ranging from 0.1-10 μm. In certain embodiments, utensil 100 exhibits hemispherical reflectance of less than 0.5%, under the same conditions. In certain embodiments, the hemispherical reflectance is less than 0.7%, less than 1%, less than 1.2%, less than 1.5%, less than 1.7%, less than 2.5%, less than 3%, or less than 4%.

In certain embodiments, the surface porosity of coating 120 is 50%, with the majority of the pores between 5-20 mm diameter. In certain embodiments, the combination of different pore sizes enables the trapping, or constraining the mobility, of both cells and macromolecular reagents such as biological ligands, thus facilitating the biological reaction of interest. In certain embodiments, the porosity of the surface contributes to its low fluorescence and/or reflectivity.

In certain embodiments, the surface of utensil 100 comprises an array of pores, wherein substantially all the pores have a similar diameter, for Example wherein the mean pore diameter is between about 20 and about 90 micrometers. In more specific embodiments, the majority of the pores are each connected to at least four other pores. In even more specific embodiments, the diameter of the majority of the connections between the pores is between about 15% and about 40% of the mean diameter of the pores.

In certain embodiments, coating 120 and/or utensil 100 exhibits a laser-induced damage threshold of 400 W/cm² or higher under conditions of a direct irradiation for 60 seconds at a wavelength of 1064 nanometers (nm), a pulse length of 1 μs, a repetition rate of 20 kHz, and a beam size (A_{eff}) on the sample of 1.8*10⁻² cm². In certain embodiments, the damage threshold under the same conditions is 200 W/cm² or higher. In certain embodiments, the damage threshold is 250 W/cm² or higher, 300 W/cm² or higher, 350 W/cm² or higher, 450 W/cm² or higher, 500 W/cm² or higher, 600 W/cm² or higher, 800 W/cm² or higher, or 1000 W/cm² or higher.

In certain embodiments, coating 120 on utensil 100 exhibits a high surface area. In more specific embodiments, the surface area is at least 100 m²/m², at least 150 m²/m², at least 200 m²/m², at least 300 m²/m², at least 500 m²/m², at least 700 m²/m², at least 1000 m²/m², at least 1500 m²/m², at least 2000 m²/m², at least 3000 m²/m², at least 500 m²/m², at least 700 m²/m², or at least 1000 m²/m².

In certain embodiments, coating 120 may be roughened. In some embodiments, utensil 100 may comprise a foil which is embossed or otherwise roughened in a reel-to-reel process and is then coated by coating 120. In certain embodiments, a roll may be coated with black coating 120 and then embossed or otherwise roughened. Methods of roughening include but are not limited to deposition of a valve metal and etching.

In certain embodiments, the surface of coating 120 and/or utensil 100 may be treated to enable its binding to a desired target (see e.g., Table 3), for example a cell, nucleic acid, protein or other biomolecule, thus enabling rapid and specific isolation of the desired target. In certain embodiments, a target biomolecule may be a covalently-attached adhesion molecule, for Example a polysaccharide. This is accomplished in some embodiments by conjugating the surface to a molecule selected from a polypeptide, a protein, an antigen, an oligonucleotide, streptavidin, and another molecule with an affinity for the desired target. In certain embodiments, the target molecule may be linked to the surface of coating 120 via a silicon-based molecule. Suitable molecules include modified versions of silane-based compounds containing 3-4 alkoxy moieties, such as ethylene tetramethoxysilane or ethylene tetraethoxysilane, wherein the modification is replacement of at least one alkoxy moiety with a substituent terminating in a reactive group, for Example an alkyl moiety terminating in a reactive group, such as sulfhydryl, ammonium, carbodiimide, or the like. Reactive groups also include cross-linking moieties, which are, in some embodiments, subsequently reacted with a bridge reagent. Cross-linking moieties include amino, hydroxyl, carboxyl and sulfhydryl reactive groups (e.g., malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio). Other sulfhydryl-reactive moieties include haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio. In various embodiments, the bridge reagent is heterobifunctional or homobifunctional, and may be attached in active form or is subsequently activated, for Example by visible light or ultraviolet irradiation; and either is or is not cleavable. In a non-limiting embodiment, the cross-linking reagent may be alkylthiol, and the bridge reagent may be a cleavable agent with two maleimido moieties. Besides the illustrated cleavable ester group, other chemically cleavable groups include dialkoxysilane, 3′-(S)-phosphorothioate, 5′-(S)-phosphorothioate, 3′-(N)-phosphoramidate, 5′-(N)phosphoramidate, and ribose. For Example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In certain embodiments, any of the following ligand coupling methods may be used: coupling with a primary antibody against specific cell surface antigens; coupling with a secondary antibody for use with a chosen primary antibody; configuring coating 120 to have specific surface functionalities for direct coupling of targets, ligands or target molecules; using streptavidin with biotinylated ligands or targets; coupling with protein A or protein G for immunoglobulin (Ig) purification and imunoprecipitation; and coupling with oligo-dT for isolation of polyadenylated mRNA.

In certain embodiments, binding may be enhanced by the porous structure of layer 120 and/or coating, by concentrating bound molecules to pore regions 123B, by applying a suitable over-coating layer 121 and/or by defining fine features and patterns to limit or conduct binding molecules to specified sites. Advantageously, coating 120 provides a higher spot resolution due to concentration of markers or pigments to pores of ceramic layer 122.

In certain embodiments, the crosslinker has a reactive electrophilic group tethered on a spacer, which covalently binds protein (e.g. antibody) molecules by binding free NH₂ and/or SH groups of the proteins. In certain embodiments, the crosslinker has a secondary amine on a spacer, which couples with —COOH or —PO₄— groups, or with linked maleimide groups. In certain embodiments, zwitterionic carboxybetaine polymer (pCB) coated substrates as used as an array surface platform to enable amino-coupling chemistry. In certain embodiments, a colloidal nitrocellulose substrate is used for target immobilization of proteins; or the conjugation may be performed using Versalinx chemistry; using nitrocellulose chemistry; or using a streptavidin moiety. In certain embodiments, a protein microarray is created using HaloTag fusion proteins.

In certain embodiments, the surface of utensil 100 may be conjugated to one or more of the following ligands: A poly-D-lysine coated surface, which may be useful for facilitating attachment of difficult-to-attach cells; A Sulfhydryl binding surface has covalently-linked maleimide groups that covalently couple to sulfhydryl groups via SH moieties. Useful for assays requiring site-directed orientation of a biomolecule, especially antibodies; A carbohydrate binding surface has hydrazide groups that covalently bind to carbohydrate groups. Useful for assays requiring site-directed orientation of a biomolecule (oxidized antibodies, carbohydrates, and glycosylated proteins) while maintaining enzymatic or immunological activity; A photo-reactive surface that covalently immobilizes biomolecules via abstractable hydrogens using UV illumination, resulting in a carbon-carbon bond. Although linkage is nonspecific and does not allow for site-directed orientation of a biomolecule, this surface may be useful for immobilization of double stranded DNA, antigens of unknown structure, and mixtures of biomolecules (e.g., cell lysates); and an amine surface that has positively charged amine groups (2×10¹³ reactive sites/cm²) that can be used for covalent immobilization via bifunctional crosslinkers.

Conjugated utensils 100 are in some embodiments particularly suitable for coated-plate assays. Sometimes referred to as “solid phase assays”, coated-plate assays require the anchoring of one of the assay components to the surface of the microplate. Coated-plate assays use wash steps to separate bound (associating) and unbound (non-associating) reagents from the well of the plate. The anchored component may be a protein, antibody, or other macromolecule, or may be an intact cell, particularly when the coating contains pores on a micrometer scale, as described herein.

In certain embodiments, utensil 100 may comprise barcodes for accurate data collection.

In certain embodiments, utensil 100 may comprise a crystallization microplate, used for high-throughput protein crystal growth and screening.

Alternatively or in addition, utensil 100 as multi-well plates may comprise multiple spots per well, which may be placed by various technologies. In certain embodiments, the exact positioning of the detection molecules in predefined patterns enables precise, accurate and reproducible detection of analytes and high density of information as well as parallel processing of assays. In certain embodiments, the pattern is created by micro-structuring that imparts chemical and physical characteristics of interest. In some embodiments the micro-structuring either (a) delimits the substance (e.g. a binding antibody) on the spotted area, for Example by creating a precise pattern of surfaces amenable to antibody attachment, or (b) directs precise functionalization and surface treatment.

In certain embodiments, antibodies or other proteins or biological molecules are placed in spots using solid pin contact printers or noncontact piezoelectric printers such as a non-contact dispenser that generates free flying droplet. In certain embodiments, the coating is heat-resistant and thus able to withstand laser scanning detection. Alternatively or in addition, the entire inner surface of the well is resistant to washing procedures. More specifically, the washing process should not alter the specific characteristics in regard of binding properties and light absorption.

In certain embodiments, utensil 100 comprises membrane-bottom plates.

In certain embodiments, utensil 100 exhibits no significant cytotoxicity as measured using L929 mouse fibroblasts in culture medium containing 10% fetal calf serum, in accordance with DIN EN ISO 10993-5. “Significant cytotoxicity” in this context refers to an amount of cytotoxicity that adversely affects cellular viability to an extent measurable in a standard toxicity assay such as DIN EN ISO 10993-5, after a 24-hour incubation in cell culture media at 37° C. In other embodiments, the toxicity of the coating, if any, is less than the level of toxicity that would decrease cell counts by 5%, 10%, 20% or 30%, after a 24-hour incubation in cell culture media at 37° C.

Alternatively or in addition, utensil 100 is adapted for use in a biological assay, for Example, utensil 100 may be configured not to elute significant amounts of cytotoxic substances when aqueous solution is incubated in its wells and not contain or elute substances that appreciably react with reagents in biological solutions, for Example peptides, nucleotides, saccharides, and lipids. In certain embodiments, utensil 100 is shaped for compatibility with cover that blocks contamination of the contents of the well. In certain embodiments, the cover reduces evaporation of the contents of the well. In certain embodiments, the cover allows the contents of the well to be incubated in a thermocycler. In certain embodiments, the inner surface of the well contains a substrate for cell adherence.

Wherever alternatives for single features such as arrangements of the metal and the dielectric material; arrangements of the substrate (if a separate substrate is present) and the coating; methods of coating the substrate, if present; types of metals and dielectric materials; levels of fluorescence, reflectance, cytotoxicity, and damage threshold; porosity size and pattern; conjugation to other molecules; surface area, etc. are laid out herein as “embodiments”, it is to be understood that such alternatives may be combined freely to form discrete embodiments of the entire composition provided herein.

Alternatively or in addition, the substrate may be coated with a discontinuous layer of coating 120. In cases wherein the substrate is hydrophobic (water repellent), and coating 120 is hydrophilic (water receptive), the pattern may be designed to have hydrophilic and hydrophobic areas in a required configuration that may enhance the functioning of utensil 100 (e.g., enable better sample positioning, control sample flow, provide optical features etc.).

In certain embodiments, coating 120 may comprise or be designed to exhibit a specified level of hydrophilicity or hydrophobicity, configured to control water flow and adherence in utensil 100 and to control ligand attachment. For Example, Table 3 schematically illustrates various levels of hydrophilicity, which may be selected with respect to the applications of utensil 100 and the biological molecules these application involve. The levels of hydrophilicity are denoted as being none, low, medium and high, clearly specific values may be determine with respect to the exact application and bound molecules.

TABLE 3
Variable hydrophilicity levels with respect to applications.

| | | Features/ | Exemplary |
|---|---|---|---|
| Hydrophilicity | Binding preference | properties | Applications |
| − (none) | Bio molecules that have | Lower binding of | Coated antigen |
| | hydrophobic domains, e.g. | IgG: appr. 200-250 ng/cm² | ELISA |
| | lipids, lipoproteins, large | | e.g. β- |
| | proteins | | Galactosidase- |
| | | | Assay |
| + (low) | Bio molecules with | protein binding | Antibody |
| | hydrophilic/hydrophobic | moderate | sandwich ELISA, |
| | properties, e.g. medium to | binding of | coated antigen |
| | large proteins such as | immunoglobuline | ELISA |
| | albumin Amphiphilic bio | (capacity): 350-600 ng | |
| | molecules such as LPS | IgG/cm² | |
| ++ (medium) | Bio molecules with | Binding of a | Antibody |
| | hydrophilic/hydrophobic | broad range of | sandwich ELISA, |
| | properties. For high binding | proteins and bio | coated antigen |
| | of IgG and other proteins and | molecules | ELISA |
| | bio molecules that have | Immunoglobuline | |
| | hydrophilic/hydrophobic | capacity: approx. | |
| | character | 600-650 ng/IgG/ | |
| | | cm2 | |
| +++ (high) | For binding of hydrophilic | | antigen ELISA |
| | bio molecules, e.g. | | |
| | glycoproteins, glycans, water | | |
| | soluble proteins | | |

In certain embodiments, coating 120 may comprise or be designed to exhibit a specified active (covalent) binding surface configured to the binding of biological molecules thereto. For Example, Table 4 schematically illustrates various types of binding surfaces, which may be selected with respect to the applications of utensil 100 and the biological molecules these application involve. The types of binding surfaces are characterized with respect to essential features thereof, detailed design may be determine with respect to the exact application and bound molecules.

TABLE 4
Various types of binding surfaces with respect to applications.

| Structure of | | | |
|---|---|---|---|
| the binding | | | |
| surface | Binding preference | Features/properties | Key Applications |
| Reactive | Covalent binding of | Immobilizing of proteins and | antigen ELISA, |
| electrophilic | bio molecules with | peptides that do not bind to | antibody sandwich |
| group | free NH₂ and/or SH | passive surfaces. | ELISA |
| tethered on a | groups, e.g. proteins, | Stable covalent bond | |
| spacer arm | peptides | formation with free NH₂ or | |
| | | SH groups via spacer arm. | |
| | | Could reduce the amount of | |
| | | bio molecule needed for | |
| | | coating vs. passive binding | |
| Secondary | Covalent coupling of | Can link molecules via the | Antigen ELISA |
| Amine on a | bio molecules with —COOH | COOH group (enables the | |
| spacer arm | or —PO₄- | detection of peptides that bind | |
| (e.g. of 2 nm) | groups | to an antibody via the NH₄ | |
| | | end). | |
| | | Spacer arm binding for | |
| | | optimal orientation | |
| Covalently | Covalently couple to | Sulfhydryl binding surface | Ideal for assays |
| linked | sulfhydryl groups via | | requiring site- |
| maleimide | SH moieties. | | directed |
| groups | | | orientation of a bio |
| | | | molecule, |
| | | | especially |
| | | | antibodies |

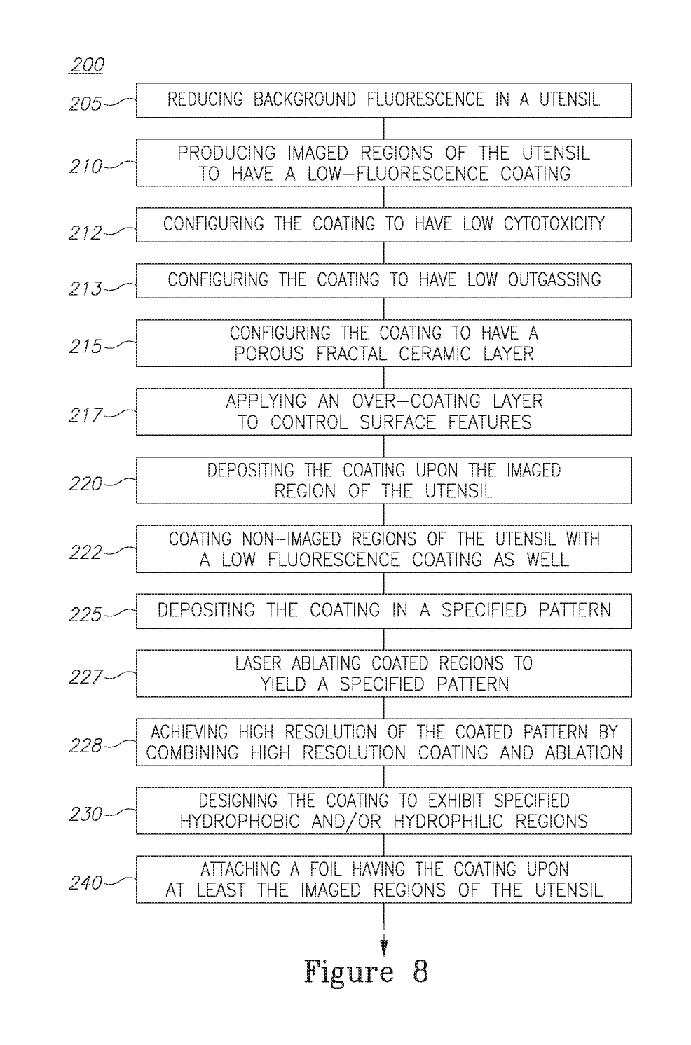

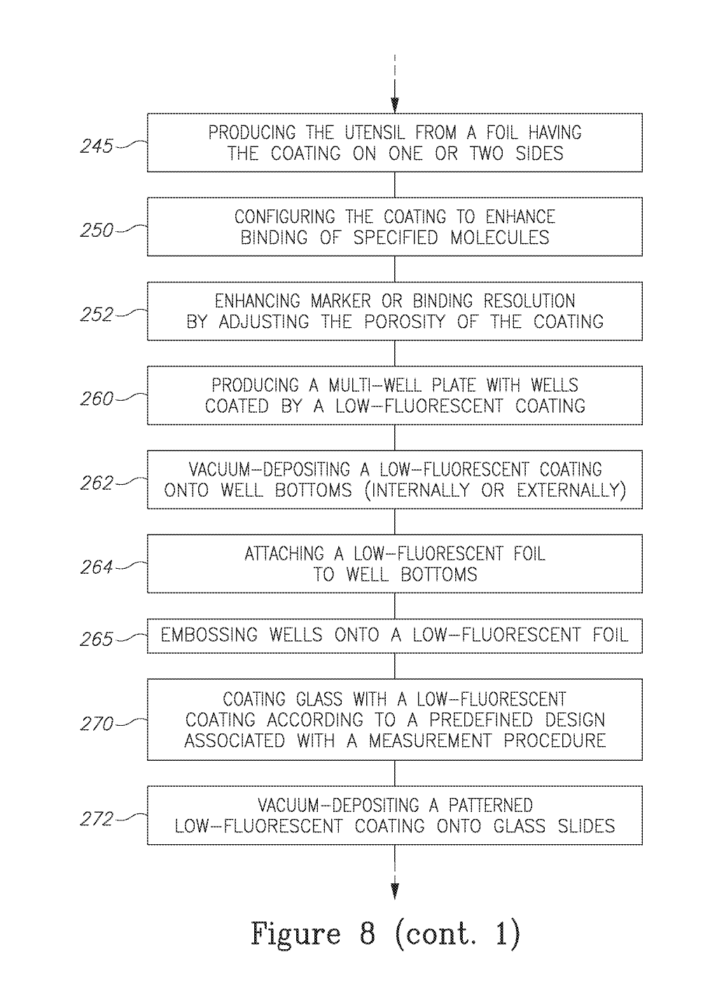

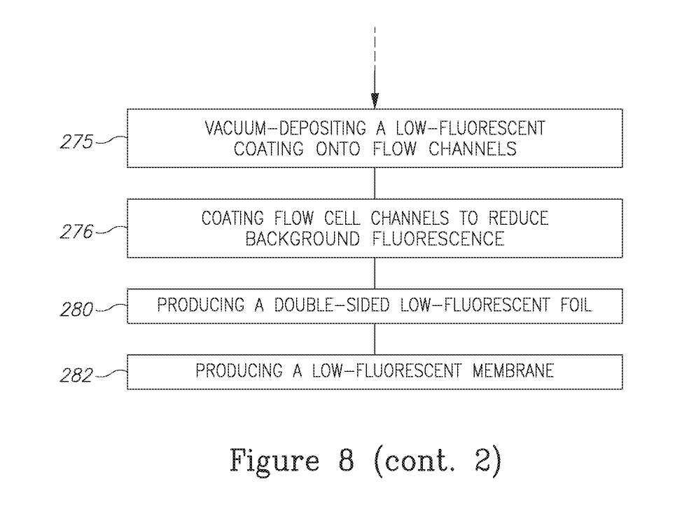

FIG. 8 is a high level schematic flowchart illustrating a method 200 according to some embodiments of the invention. Method 200 comprises reducing background fluorescence in a utensil (stage 205) by producing at least an imaged region of the utensil to have a coating (stage 210) comprising at least one porous fractal ceramic layer deposited upon at least one metal layer.

The ceramic layer may be produced by reactive vapor deposition and comprise at least one oxide of: aluminum, titanium, tantalum, niobium, zirconium, silicon, thorium, cadmium and tungsten. The coating may be configured to exhibit hemispherical reflectance of less than 2% over a range of wavelengths ranging from 0.1-10 μm. The coating may be configured to have a porous fractal ceramic layer deposited upon a metal layer (stage 215). In certain embodiments, method 200 comprises configuring the coating to exhibit low cytotoxicity (stage 212) and/or configuring the coating to exhibit low outgassing (stage 213). In certain embodiments, method 200 comprises applying an over-coating layer to control surface features (stage 217).

The low fluorescence coating may be deposited upon at least the imaged region of the utensil (stage 220), optionally upon non-imaged regions of the utensil (stage 222), and optionally according to a specified pattern designed according to a microscopy target (stage 225). The specified pattern may be designed with respect to a specified hydrophobic/hydrophilic character of the pattern (stage 230), related to required fluid behavior in the utensil and/or related to binding requirements with respect to biological or other molecules. The patterned coating may be produced by laser ablation of deposited coating (stage 227). In certain embodiments, method 200 comprises achieving high resolution of the coated pattern by combining high resolution coating and ablation (stage 228).

In certain embodiments, the imaged region may comprise any of the following: a well bottom in a multi well plate, a foil or regions thereof, an embossed foil or regions thereof, a continuous array tape, a foil metal insert, a lab-on-a-foil, DNA or protein microarrays, a region on a glass slide, a flow channel in a flow cell, a microscope pad or regions thereof, microbead, membrane or regions thereof, biochip and other life science products used in florescence measurements.

In certain embodiments, method 200 further comprises attaching a foil having the coating upon at least the imaged region of the utensil (stage 240) and/or producing the utensil from a foil having the coating on at least one of its sides (stage 245). In certain embodiments, the foil may be coated on both sides and/or be used a s a membrane.

In certain embodiments, method 200 further comprises configuring the coating to enhance binding of specified molecules (stage 250). In certain embodiments, method 200 comprises enhancing marker or binding resolution by adjusting the porosity of the coating (stage 252).

In certain embodiments, method 200 further comprises configuring the coating to have a specified hydrophobic/hydrophilic character (stage 230).

In certain embodiments, method 200 further comprises producing a multi-well plate with wells coated by a low-fluorescent coating (stage 260), for Example by vacuum-depositing a low-fluorescent coating onto well bottoms (internally or externally) (stage 262), attaching a low-fluorescent foil to well bottoms (stage 264) and/or embossing wells onto a low-fluorescent foil (stage 265).

In certain embodiments, method 200 further comprises coating glass with a low-fluorescent coating according to a predefined design associated with a measurement procedure (stage 270), for example by vacuum-depositing a patterned low-fluorescent coating onto glass slides (stage 272).

In certain embodiments, method 200 further comprises coating vacuum-depositing a low-fluorescent coating onto flow channels (stage 275) to coat flow cell channels to reduce background fluorescence (stage 276).

In certain embodiments, method 200 further comprises producing a double-sided low-fluorescent foil (stage 280) and/or producing a low-fluorescent membrane (stage 282).

### Experimental Section

### Preparation of Coating

### Example 1

### Manufacturing of Coated Foils

A coated foil (referred to in the following as “Type I”) was produced by evaporating aluminum onto a substrate of clean aluminum foil, held at a temperature of 300° C., by thermal resistive evaporation, in an anhydrous atmosphere of nitrogen at a pressure of between 2×10⁻³ and 5×10⁻³ torr, and oxygen at a pressure between 2×10⁻⁴ torr and 5×10⁻⁴ torr. The deposition rate was about 300 angstroms/second, and the coating was between 3-20 microns in thickness. The Al/Al₂O₃ layer exhibited a fractal-like structure with a cauliflower-like morphology. The “cauliflower heads” (largest recurring units) were about 2 microns across. The “florets” (smallest recurring units) were about 0.2 microns across, so that the surface was self-similar at least on a distance scale from 0.2 microns to 2 microns. This was confirmed by the visual appearance of the surface. The Al/Al₂O₃ surface was black matte (diffusely reflective), showing that this surface had a fractal-like structure on the length scale of the wavelengths of visible light. Thus, aluminum foil was produced, having an Al/Al₂O₃ layer deposited on one or both sides thereof.

A coated foil (referred to in the following as “Type II”) was produced according to a similar procedure, except that the oxygen concentration was 8-10×10⁻³ torr.

A coated sample (referred to in the following as “Type III”) was produced according to a similar procedure as “Type II”, except that the coating was thicker, namely about 250 microns.

A coated sample (referred to in the following as “Type IV”) was produced according to a similar procedure as “Type I”, except that the coating was 5-7 microns and weighed 1.4-3.2 mg/cm³.

A coated sample (referred to in the following as “Type V”) was produced according to a similar procedure as “Type I”, except that the coating was 3-5 microns and weighed 0.7-1.1 mg/cm³.

A coated sample (referred to in the following as “Type VI”) was produced according to a similar procedure as “Type I”, except that the coating was 4-7 microns and weighed 1.1-1.6 mg/cm³.

It is noted that these specific characteristics are not limiting coating 120 but are merely presented as examples used for the measurements. Variations of the production process yield many other types of coatings, sharing the fractal porous ceramic layer and general characteristics but having different thicknesses and weights, which are likewise applicable under the present disclosure.

### Example 2

### Characterization of coatings

Coated foils were characterized by electron microscopy. A Type II sample, illustrated in FIGS. 3C and 3D, contained no pores in the micro-scale range and small pores in the nano-scale range (no more than 1 micron in diameter). A thicker coating of 50-100 microns (intermediate between Type II and Type III), illustrated in FIGS. 3E and 3A, contained large pores in the micro-scale range (up to 10 microns in diameter) and large and medium pores in the nano-scale range.

### Reduced Fluorescence

### Example 3

### Fluorescence of Coatings Under Conditions of Ultraviolet Irradiation

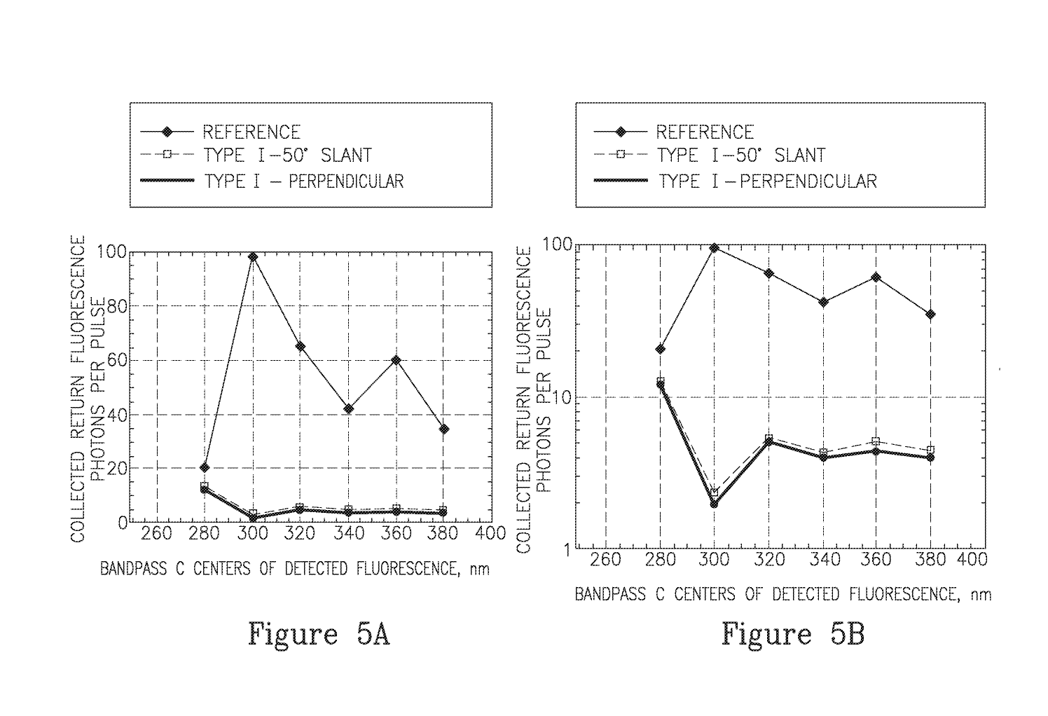

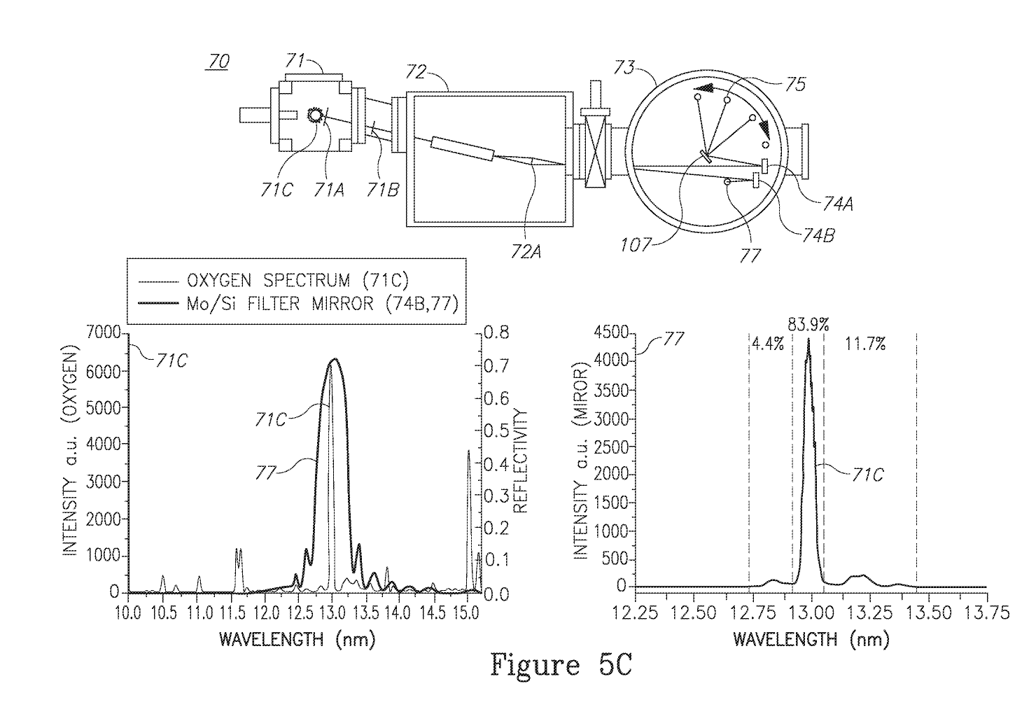

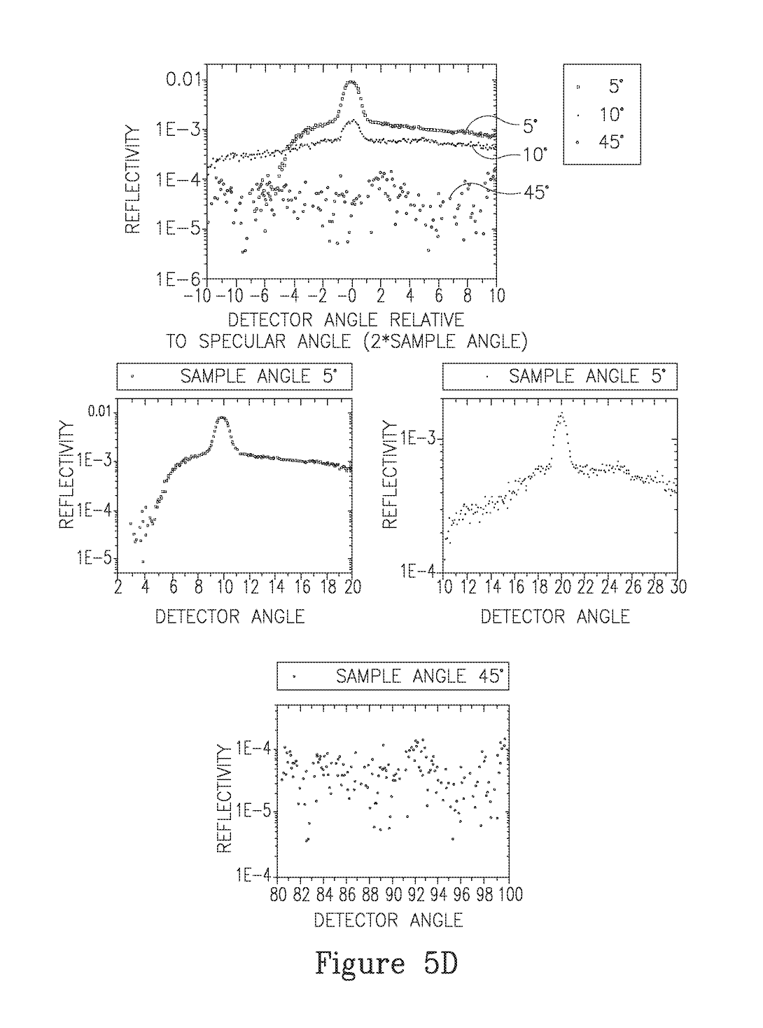

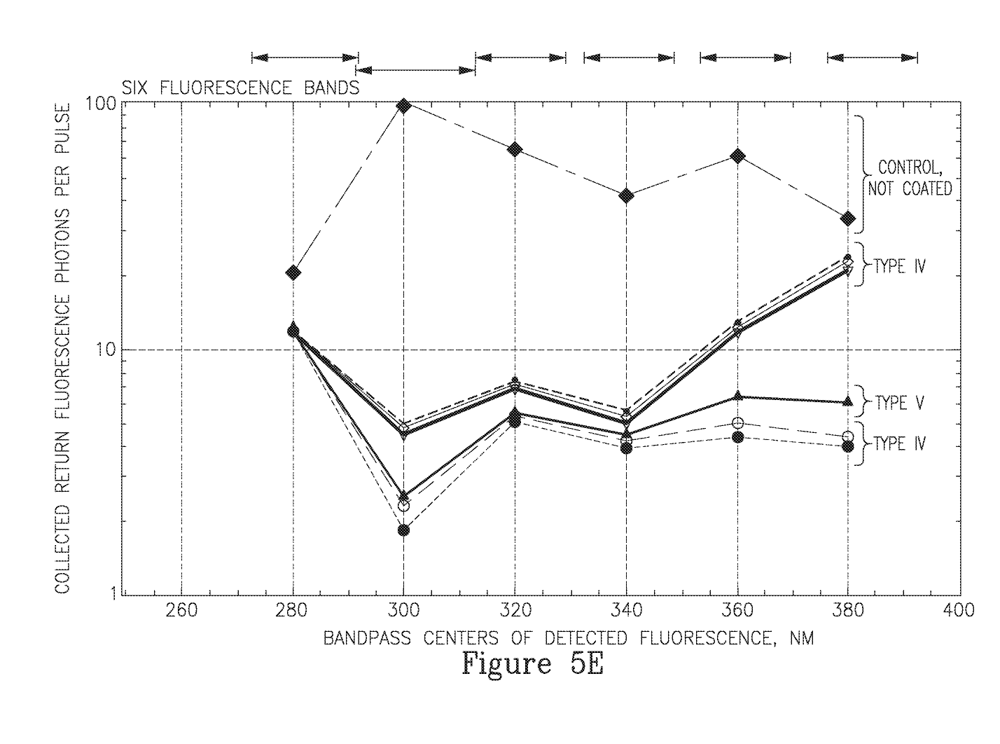

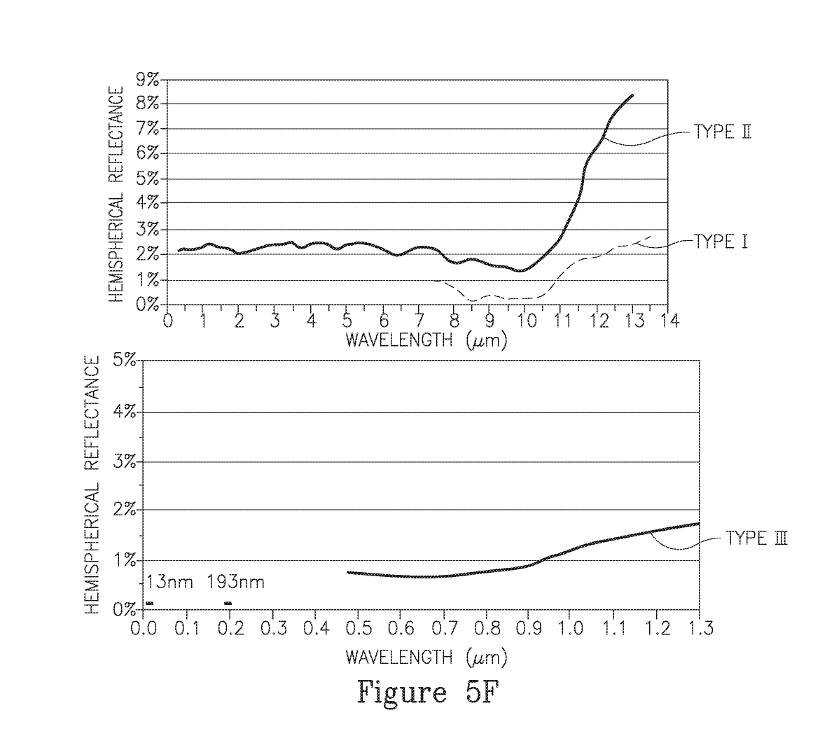

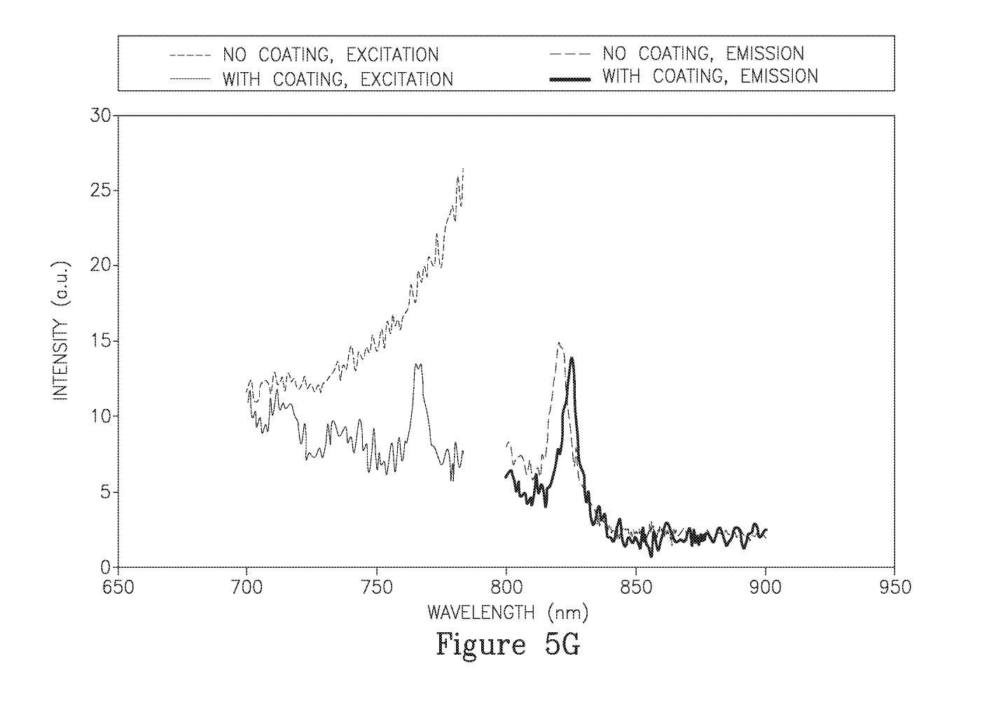

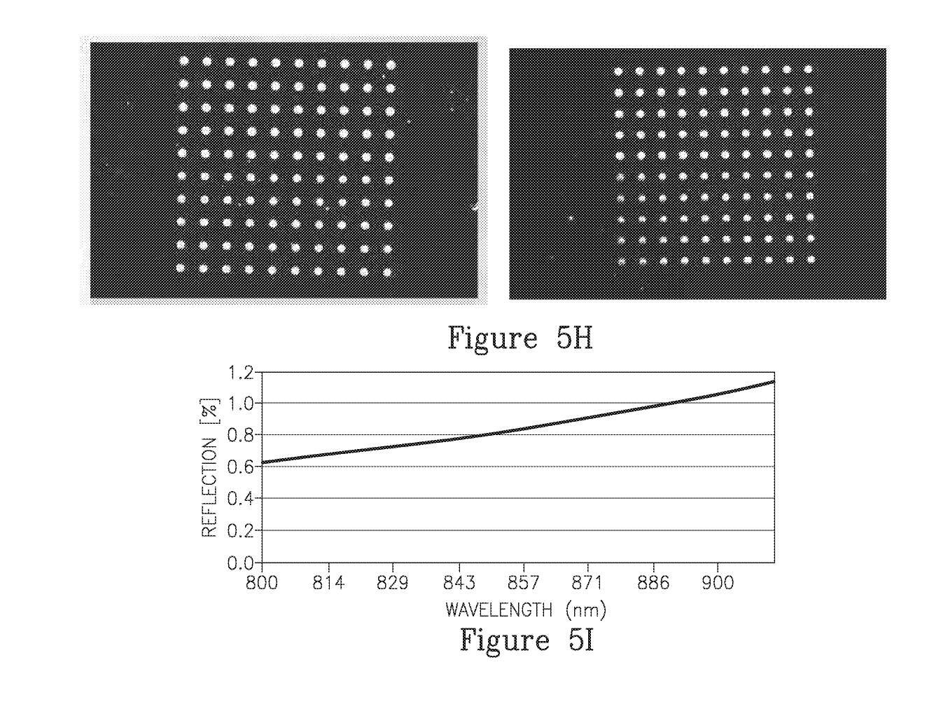

To measure fluorescence, coated foils were irradiated with a 248.6-nm laser that generates 2-4×10¹² monochromatic photons per pulse, in a pattern that time averages over ˜20 pulses onto an elliptical area with major axis of ˜7 mm and a minor axis of ˜5 mm. The ellipse was empty in the middle, with the illumination lying in ˜ a 1.5 mm thick bright ring that rotates slowly in orientation, per pulse, forming a full rotation in about 12-15 pulses. Integrated, net illumination was a circular pattern with ˜2 mm bright outer ring edge and a 7 to 8 mm outer diameter. The illumination time was ˜40 microseconds on-time and 300 milliseconds off-time. Measurements were generally 301 pulses in duration at 3 Hz, taking ˜100 seconds total time. Nominal six detection bandpasses covered 280, 300, 320, 340, 360 and 380 nm, at a 20 to 14 nm Full Width Half Maximum (FWHM) resolution. FIGS. 5A-5G present fluorescence measurements of coating 120, according to some embodiments of the invention.

FIGS. 5A and 5B illustrates results of fluorescence measurements for the Type I coating. FIG. 5A illustrates the result in a linear scale, FIG. 5B illustrates the result in a logarithmic scale. Three Type I coatings were produced on a 4 mm aluminum substrate (309-11) and measured in a perpendicular direction to the laser beam and telescope mirror (averaged results indicated as “Type I—Perpendicular”). These were measurements of direct backscatter. Three similar samples were measured with the sample tilted about 50° off the vertical in one axis to examine diffuse return (averaged results indicated as “Type I—50° slant”). The laser was targeted to the approximate center of the coated piece. As reference and control, a standard black anodized optical erector set part from ThorLab, Inc, was measured in the same inclinations.

Evidently, there was very little difference between the perpendicular and tilted measurements. The optical erector set part exhibited a much stronger fluorescence than either of the two coatings.

### Example 4

### Extreme Ultraviolet (EUV) Reflectance of Coatings

Using an extreme ultraviolet (EUV) source, reflectivity measurements were conducted on a Type I-coated foil at a center wavelength of 12.98 nm. The measurement procedures and results are described below. All angles described in this report are grazing angles (relative to the sample surface).

FIG. 5C shows the experimental setup. The EUV radiation was generated in source chamber 71 using a laser produced plasma 71C from a gas puff target. The spectral characteristics of the source depend on the employed target gas. For the measurements, oxygen was used as the target material. The light emitted by source 71C passed through a 150 μm slit aperture 71A placed approximately 2 mm behind plasma 71C and a Zirconium filter 71B which blocked higher wavelength components of the spectra. In optics chamber 72, an optics 72A in Kirkpatrick-Baez arrangement images slit 71A onto a sample 107 placed in the middle of an experimental chamber 73 via a Mo/Si multilayer mirror 74A. Mirror 74A worked as a spectral filter in the EUV spectral region (blocking out-of-band radiation) and was adapted to the emission line of oxygen at 12.98 nm. The reflectometer placed in experimental chamber 73 consists of two independent rotary stages for sample holder and detector diode 75, respectively. The beam diameter on sample 107 (perpendicular to the incident radiation) is approximately 300 μm in the horizontal direction and 500 μm in the vertical direction. For monitoring of intensity fluctuations of plasma source 71C, a second EUV photodiode 77 was used as a reference monitor. It was illuminated by a second filtering multilayer mirror 74B with the same specifications as main mirror 74A. The two graphs at the bottom of FIG. 5C characterize radiation source 71C and filter mirrors 74B as measured at reference detector 77. Additional measurement parameters are: detector scans at 5°/10°/45° sample angle, at −10° to +10° relative to specular angle and at an angular range of two times the sample angle, with an angular detector step size of 0.1°. Results are averaged over 25 EUV pulses/measurement point. Angular accuracy is <1°, angular repeatability is <0.1°, absolute accuracy of reflectivity is <5% and absolute repeatability of reflectivity is <0.5%.

FIG. 5D shows the measurement results. Three measurements were conducted to analyze the EUV light-scattering properties of the sample. For these, the angle of incidence of the light onto the sample was set to 5°, 10°, and 45° grazing angle (angle to sample surface), respectively. The results for these three angles are present in FIG. 5D together and separately. For each of these positions, a detector scan was made, centered on the angle of specular reflectance with a range of −10° to +10°. All the measurements were background corrected. The data recorded for 45° sample angle represents the noise level of detector 75. At this sample angle, either all radiation impinging on sample 107 was absorbed, or the reflected/scattered light was too weak to be detected with the equipment. At 5° and 10° sample angles, the specular reflectance of the measurement beam can be seen as peaks in the middle of the respective datagrams. These peaks have a reflectivity of approximately 1% and 0.1% for 5° and 10° sample angle respectively. The flanks of these peaks correspond to the scattered light. Their asymmetry stems from the fact that at small angles of incidence, the scattered light is blocked by the sample itself. Hence, sample 107 which is representative of some embodiments of coating 120 for utensil 100, has very low scattering characteristics.

### Example 5A

### Fluorescence of the Coating at Various Fluorescence Bands

Fluorescence of various coatings 120 at various slant angles were measured using a Targeted UV Chemical, Biological and Explosives (TUCBE) instrument which contains a 248.6 nm hollow cathode discharge laser that generates an output pulse of 40 to 60 μsec duration, at repetition rates of 1 to 20 Hz. The output intensity is variable over a modest range; most of the measurements were performed with 2 to 3 μJoules/pulse [2 to 4×10¹² photons], at 3 Hz. The instrument contains six narrow band pass limited PMT detectors, covering the region between 270 and 410 nm. The detectors have gate-synchronized on-time with the laser pulse and also make a pre-sequential ambient, non-laser light measurement with the same integration time as that selected for the laser fluorescence signal measurement.

The instrument optics provide optimum performance in signal detection at a nominal distance of 68 inches (1.72 m), but the detection signal peak is not narrow; best response covers a distance range of about 4 inches (10 cm). Off-geometry measurements were performed nominal 50° off vertical, with the offset plane moving further away from the instrument.

FIG. 5E illustrates the six fluorescence bands and a comparison of the collected return fluorescence photon per pulse for three types of coatings (IV, V, VI), each under nearly perpendicular measurement and in a 50° slant. The coated parts are compared to a black anodized aluminum part, which exhibits significantly higher fluorescence than the coated parts over all six bands. For the three types of coatings 120 there was generally little to no difference in detected fluorescence between the sample perpendicular to the laser beam or tilted back about 50°. The fluorescent yields are well below 10⁻¹⁰ while the uncoated part exhibited fluorescence fluxes which were one to two orders of magnitude higher than the coated parts.

### Example 5B

### Hemispherical Reflectance of Foils and Coatings at Different Wavelengths

FIG. 5F illustrates the hemispherical reflectance at different wavelengths of foils with various coatings. Substances exhibited very low reflectance generally around 2% up to 10 μm and below 1% up to 0.9 μm. Type II exhibited higher hemispherical reflectance at wavelengths of over 11 μm. Type III exhibits low hemispherical reflectance at shorter wavelengths.

### Example 6

### Fluorescence and Absorbance Measurements of a Blue-Fluorescing Part

Coated aluminum rigid plates exhibited a bluish appearance, prompting experiments to confirm that they exhibited low reflectance and low fluorescence, similar to the other tested coatings and coated items. A fluorometer was used to measure the excitation (absorbance) and emission spectrums of the coated part in comparison to blank, uncoated aluminum.

FIG. 5G illustrates the comparison of excitation and emission spectra with and without coating 120. Excitation of the coating was significantly lower than excitation of the blank aluminum part and emission was slightly lower. It is noted that the narrow peak appearing at 826 nm in both emission spectrums of blank aluminum reference and coated part is an instrumental effect formed due to the excited state intensity.

FIG. 5I illustrates the hemispherical spectral reflectance of the sample, which exhibited reflectance of less than 1% at 840 nm. In conclusion, the tested part did not exhibit any deviation of the reflectivity from the specification. Moreover, they were shown to be non-fluorescent.

### Porosity of the Coating

### Example 7

### Pore Distribution and Other Physical Characteristics of Coatings

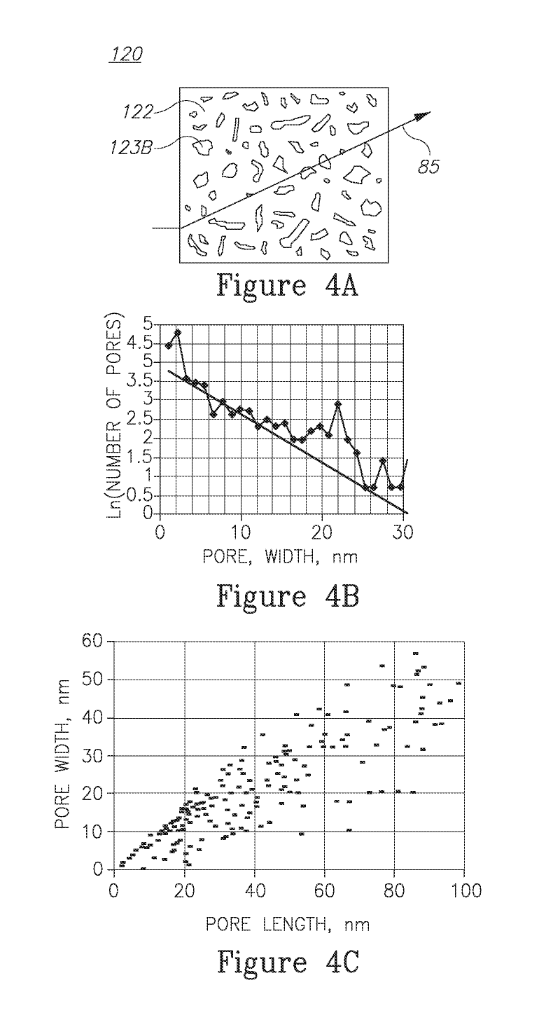

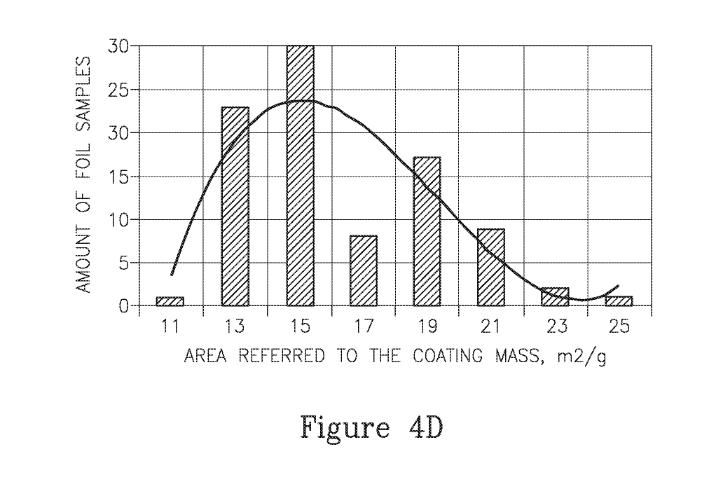

FIGS. 4A-4D illustrate the porosity and fractal structure of coating 120, according to some embodiments of the invention. FIG. 4A schematically illustrates the surface of ceramic layer 120, FIG. 4B illustrates a distribution of pore width along scanning line 85 that is illustrated in FIG. 4A and FIG. 4C illustrates the distribution of pore widths and length. A foil having a thickness (D) of 63.5 μm was subjected to single-side deposition, using coating 120 depicted in FIGS. 3C and 3D. The geometry of the surface layer pores was determined by scanning electron microscopy (SEM) to a depth of 0.5 μm as described in FIG. 4A. The pore width exhibited the distribution shown in FIG. 4B and the width vs. length distribution data shown in FIG. 4C.

FIG. 4D illustrates a distribution of a specific area of coating over ca. 90 samples according to some embodiments of the invention. In the illustrated example, the median value of the initial specific area (Q_{coat}, or effective area of the coating per unit of its mass) of coated foils 120 was 15 m²/g. If the substrate mass was taken into account, the values of specific area were smaller. For Example, if the foil thickness D=63.5 μm, then the value of Q_{coat} is 6.5 m²/g.

The porosity of the coating was assessed by the oil absorption method, as presented in Table 5. In one of the experiments, nine zones were measured on each of three samples with respect to their thickness, volume, mass and mass and volume of absorbed oil in these regions (for hydrophobic ceramic layers 122 in some embodiments of the invention). The resulting porosity is between 25 and 35%.

TABLE 5
Porosity measurements by oil absorption.

| | Uncoated | Sample | | | |
|---|---|---|---|---|---|
| | foil | No. 1 | No. 2 | No. 3 | Unit |
| Coating thickness | | | | | |
| measured at: | | | | | |
| zone 1 | | 16.5 | 21.0 | 23.0 | μm |
| zone 2 | | 15.0 | 21.0 | 21.5 | μm |
| zone 3 | | 15.0 | 20.0 | 21.5 | μm |
| zone 4 | | 19.0 | 24.0 | 25.0 | μm |
| zone 5 | | 17.5 | 23.5 | 24.5 | μm |
| zone 6 | | 17.0 | 23.0 | 24.5 | μm |
| zone 7 | | 17.0 | 21.5 | 23.0 | μm |
| zone 8 | | 15.5 | 21.5 | 22.5 | μm |
| zone 9 | | 15.0 | 21.5 | 22.0 | pm |
| Average thickness | | 16.4 | 21.9 | 23.1 | pm |
| Total foil & oil mass | 676.4 | 823.1 | 851.0 | 858.5 | mg |
| Mass of uncoated foil | 669.0 | 805.0 | 820.5 | 826.7 | mg |
| Mass of absorbed oil | 7.4 | 18.1 | 30.5 | 31.8 | mg |
| Volume of oil | 8.51 | 16.55 | 30.80 | 32.30 | mm³ |
| Volume of coating | 0 | 65.56 | 87.56 | 92.24 | mm³ |
| Porosity | 0 | 25.2 | 35.2 | 35.0 | % |

These measurements illustrate the porosity of ceramic layer 122 (FIG. 4C, Table 5), its extended surface area (FIG. 4D) and its fractal character by exhibiting self-similarity as a power law dependency over a range of pore sizes (FIG. 4B).

In certain embodiments, the densities of some coating varieties ranged between 1.8 and 2.3 g/cm³. The composition of coating 120 ranged in some of the samples around 50% Al, 50% Al₂O₃ (±10%). Elemental composition of several of the samples, according to some embodiments, is presented in Table 6.

TABLE 6
Elemental composition of several of the samples

| Element, | Sampe Number | | | | | |
|---|---|---|---|---|---|---|
| atomic % | Old | 978 | 1026 | 1030 | 1098 | 1104 |
| Aluminum | 66.38 | 68.82 | 63.22 | 65.79 | 65.7 | 63.4 |
| Oxygen | 30.43 | 28.171 | 33.81 | 32.04 | 34.3 | 36.6 |
| Carbon | | 3.011 | 2.971 | 2.17 | | |
| Nitrogen | 1.18 | | | | | |
| Phosphorus | 1.79 | | | | | |
| Argon | 0.22 | | | | | |

The inventors have discovered that an atomic ratio of aluminum to oxygen atoms which is between 0.66-4.8 did not cause cytotoxic effects (see below). In certain embodiments, ceramic layer 122 and/or coating 120 and/or a top portion thereof may exhibit such an atomic ratio to avoid being cytotoxic. In certain embodiments, coating 120 may comprise a metal core covered by an oxide layer which renders coating 120 non-toxic.

### Non-Cytotoxicity

### Example 8

### Cytotoxicity Measurements of Coated Samples

The aim of this part of the study was to find out if cytotoxic substances are extracted from the study materials, a titanium sheet with a ceramic coating, namely the coating depicted in FIGS. 3C and 3D, with cell culture medium containing 10% fetal calf serum. The test was performed according to DIN EN ISO 10993-5 as the growth inhibition test with L929 mouse fibroblasts. The growth inhibition test is designed to ascertain the presence of extractable cytotoxic substances. The growth rate of mammalian cells is significantly decreased in the presence of toxic substances. Usually, the growth rate is determined by comparing the cell number or the protein content of cells at different time intervals. In the present quantitative cytotoxicity assay, proliferating cell cultures were exposed to a dilution series of material extracts, and the growth rates were determined in comparison to positive (e.g., culture medium with 10% Dimethylsulfoxide) and negative controls (non-extracted culture medium).

Since there is a linear relationship between cell number and protein concentration under the conditions of this cytotoxicity assay, the number of viable cells, as assessed by their protein concentration, is indicative of the relative cytotoxicity of the tested extract concentration.

The test system was based upon the following standards: DIN EN ISO 10993-5, 2009, Biological evaluation of medical devices—Part 5: Tests for in vitro cytotoxicity; DIN EN ISO 10993-12, 2008. Biological evaluation of medical devices—Part 12: Sample preparation and reference materials; DIN EN ISO 10993-1, 2003, Biological evaluation of medical devices—Part 1: Evaluation and testing; USP 31, 2008, Chapter 87—Biological reactivity tests, in vitro.

Chemicals and Materials used in the study: Cell culture plates: 96 well microliter plates (Corning Lot No. 22509021); Cell culture dishes: 100 mm Ø (Corning Article No. 3100); Trypsin/EDTA: 0.05% Trypsin-EDTA (Gibco Lot No. 670380); DMSO: Dimethylsulfoxide, >99.5% (Serva Lot No. 090153); Ethanol:(technical grade) 99% denatured by the addition of 1% Butanon and other Ketons, 10 ppm Denatonium-benzoate (BfB: Federal Monopoly, Offenbach); Crystal violet stain:0.1% in water, w/v (Crystal violet, LMK Lot No. 7406); Triton×100 solution: 0.2% in water, v/v (Triton×100, LMK Lot No. 7407); Cell culture medium: DMEM (Dulbecco's modified Eagle Medium, LMK Lot No. 7421) containing: 10% (v/v) fetal calf serum (FCS, Gibco Article No. 10270-106), 584 mg/1 L-Glutamine (PAA Article No. M11-006), 50 mg/l L(+) Ascorbic acid, Na-salt (Merck Article No. F774427), 140.000 U/l Penicillin (Serva 31749), 140 mg/l Streptomycin (Serva 35500); PBS: Phosphate buffered Saline solution (PBS, LMK Lot No. 7293) containing: 0.004 M KH₂PO₄, 0.011 M Na₂HPO₄*2 H₂O, 0.003 M KCl, and 0.119 M NaCl, equilibrated to pH 7.2 with NaOH.

The growth inhibition test used conforms to the guidelines and standards set forth in the aforementioned DIN EN ISO and USP 31 documents. L929 cells were obtained from the American Type Culture Collection, Rockville, Md., USA (ATCC no. CCL1, NCTC clone 929, connective tissue mouse, clone of strain L), referred to as L929 mouse fibroblasts. A cell bank containing L929 mouse fibroblasts was kept at −196° C. Moreover, L929 cells were permanently cultivated using standard cell culture techniques (incubation at 37±1° C. in humidified air containing 5% CO₂). The cultivated cells were regularly controlled for cell growth and absence of mycoplasmas.

For the growth inhibition test, proliferating L929 mouse fibroblasts (passage 596) from stock cultures were used. They were trypsinized carefully, diluted with cell culture medium (DMEM) to a concentration of 40.000 cells per ml. From this suspension, 100 μl were pipetted into the inner wells of 96-well microtiter plates (equivalent to 1.3×10 cells/cm²). The outer wells of the plates were filled with DMEM. All plates were incubated in an incubator at 37±1° C., with 5% CO₂ and approximately 95% of relative humidity. Four hours after seeding the cells into one complete plate, they were stained with crystal violet for the determination of the initial cell density (at t=0). At the same time, the wells of the other plates were filled with the dilution series of the test materials and controls. Ten replicates of every dilution step of each extract or sample were tested in different wells. Thereafter, the plates were incubated for 72±2 h at 37±1° C., in a humidified atmosphere containing 5% CO₂.

At the end of the incubation period (t=72 h), the cells were studied under an inverted microscope at 100× magnification for their condition. Thereafter, the biological end point was determined by staining with crystal violet according to the following protocol: supernatant medium was removed; cells were washed twice with PBS; cells were fixed with 100 μl Ethanol, 99% for 10 min; ethanol was removed; cells were stained with 100 μl of crystal violet solution for 25 min; cells were washed four times with distilled water to remove remaining stain; cells were covered with 50 μl of Triton×100 solution; 10 min later, optical extinctions were measured at 570 nm.

The following tests were used as controls for the measurements:

Negative control I: Cell culture medium (DMEM+10% FCS), not incubated
Negative control II: Cell culture medium (DMEM+10% FCS), incubated for 24 hours under the same conditions as the extracts
Positive control: Dilution series of Dimethylsulfoxide (10−3−1−0.3% DMSO)

Table 7 summarizes the results of the quantitative cell growth inhibition test. None of the extract-concentrations of the coated sample exhibited any cytotoxic reaction. All values are presented as mean values out of ten together with the corresponding standard deviation (SD). Table 8 summarizes the results of a microscopic evaluation, according to the following definition of score values (according to USP—Universal Sample Processing Methodology): 0—no reactivity, Discrete intracytoplasmatic granules; no cell lysis; 1—slight reactivity, Not more than 20% of the cells are round, loosely attached, and without intracytoplasmatic granules; occasional lysed cells are present; 2—mild reactivity Not more than 50% of the cells are round and devoid of intracytoplasmatic granules; no extensive cell lysis and empty areas between cells; 3—moderate reactivity Not more than 70% of the cell layers contain rounded cells or are lysed, Nearly complete destruction of the cell layers; 4—severe reactivity.

TABLE 7
Results of growth inhibition.

| | Growth inhibition (%): Mean | | | | | |
|---|---|---|---|---|---|---|
| | value ± Standard deviation | | | | | |
| | Concentration of dilutions (v/v) | | | | | |
| | 100% | 30% | 10% | 3% | 1% | 0.3% |
| Coated sample | 7 ± 9 | 2 ± 6 | 3 ± 6 | 2 ± 6 | — | — |
| Negative control I | −2 ± 6 | — | — | — | — | — |
| Negative | 5 ± 6 | — | — | — | — | — |
| control II | | | | | | |
| Positive control | — | — | 105 ± 1 | 84 ± 4 | 16 ± 7 | −8 ± 11 |

The coated sample is better at low concentrations than the positive control as it exhibits lower growth inhibition (%). At high concentrations the coated sample has a comparable value to the negative incubated control.

TABLE 8
Results of microscopic evaluation:

| | Score values as defined below | | | | | |
|---|---|---|---|---|---|---|
| | (after 72 hours of cell culture) | | | | | |
| | Concentration of dilutions (v/v) | | | | | |
| | 100% | 30% | 10% | 3% | 1% | 0.3% |
| Coated sample | 0 | 0 | 0 | 0 | — | — |
| Negative control I | 0 | — | — | — | — | — |
| Negative control II | 0 | — | — | — | — | — |
| Positive control | — | — | 4 | 3 | 0-1 | 0 |

The coated sample is better at low concentrations than the positive control as it exhibits no reactivity over all concentrations, while the positive control causes at least some destruction of the cell layers. The coated sample shows no reactivity over the whole range of concentrations (3-100%).

In conclusion, due to the high sensitivity of the mouse fibroblast growth inhibition test, it is assumed that a mean growth inhibition of up to 30% does not indicate a significant risk of cytotoxicity. Based upon the observed results and under the test conditions chosen, the coated sample was considered to have no cytotoxic effects in all extracts in the growth inhibition test with L929 mouse fibroblasts.

### Example 9

### Outgassing Measurements of Coatings

The CVCM (Collected Volatile Condensable Material) and RML (Recovered Mass Loss) levels of a coated foil as utensil 100, having coating 120 depicted in FIGS. 3C and 3D, were measured. Due to the high specific surface area of the coatings, they exhibit enhanced absorption of water vapor in an ambient atmospheric environment, leading to a somewhat elevated level of total mass lost (TML), but when the water vapor regained (WVR) is taken into consideration, the actual RML is very low. Mass spectroscopy measurements of the out-gassing from coatings obtained from Residual Gas Analysis (RGA) measurements do not show desorption of components other than water vapor.

Table 9 summarizes the results of the outgassing measurements. The sample was baked prior to the test for 2 hours at 1800° C. and then was tested as per ASTM E-595. The data indicate clearly that the mass loss of the coating is due almost entirely to absorbed water vapor. The standard 24-hour WVR test yielded a WVR 0.31, but when the time was extended to 72 hours, the RML was reduced to 0.023. The reason for this is that the morphology of the coatings slows down the processes of water loss and regain.

TABLE 9
Outgassing characteristics of the coating.

| | Standard 24 hr WVR | 72 hr WVR |
|---|---|---|
| CVCM (%) | 0.0* | 0.0* |
| TML (%) | 1.53 | 1.53 |
| WVR (%) | 1.22 | 1.506 |
| RML (%) | 0.31 | 0.023 |

The CVCM was too low to be measured by standard instrumentation

In certain embodiments, coating 120 is configured to exhibit very low levels of outgassing, which may comply to any medical or laboratory standard. For example, the TML value may be less than 1.0% and/or the CVCM value may be lower than 0.1%. Coating 120 was found to comply with the ECSS-Q-70-02A Standard where the requirement is RML lower than 1%. In certain experiments, the absolute mass of the absorbed water vapor was very small, due to the fact that the coatings are very thin and have a very low mass. The absolute TML was of the order of 0.04 mg/cm², and the RML was of the order of 0.01 mg/cm². In certain configurations, below 100° C., outgassing/loss of water vapor proceeded very slowly, and did not increase until reaching temperatures above 115° C. which are not encounter in many of the medical and laboratory uses. In certain embodiments, coating 120 exhibits CVCM of less than 0.03% and/or TML of less than 0.7% or less than 0.07%. In certain embodiments, coating 120 exhibits CVCM of 0.001% or less and RML of 0.2% or less. In certain embodiments, coating 120 exhibits CVCM equal to 0%.

The very low outgassing rates with respect to other products (e.g., glass slides coated with nitrocellulose) have several advantages, including reduced error rate due to less interference by emitted vapor and gases and reduced interaction of emitted vapor and gases with the samples. Furthermore, the low outgassing rate of the disclosed coatings increases significantly the shelf life of utensils 100, creating thus a significant advantage. Another advantage is the inorganic nature of coating 120 with respect to other products using organic coatings which may interact with the samples and are less stable.

### Laser-Induced Damage

### Example 10

### Laser-Induced Damage Threshold (LIDT) Tests of Black Coatings

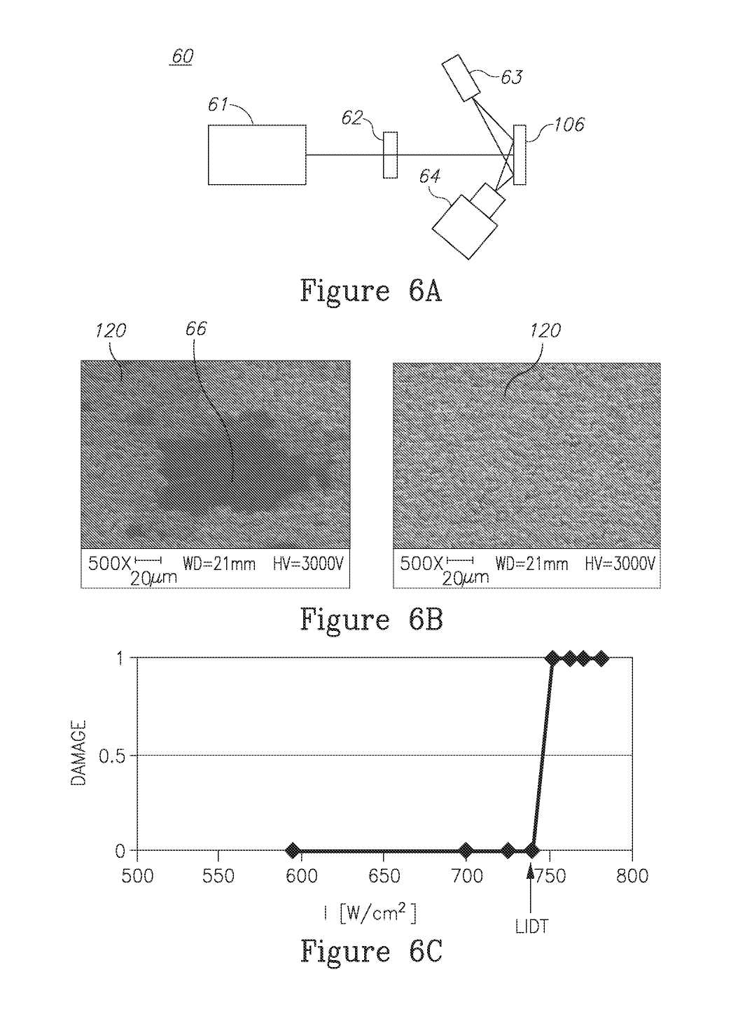

The stability of coatings 120 with respect to laser ablation was studied in order to check the possibility of patterning coating 120 using laser ablation as well as a general indication of the stability of the coating. The following coatings 120 were tested: (i) Stainless steel coated with Type I #333, thin substrate (0.65 mm); (ii) Stainless steel coated with Type II #332, thin substrate (0.65 mm); (iii) Aluminum coated with Type II #315, thick substrate (3.00 mm) The tests were carried out for different exposure times at the wavelengths 1064 nm and 532 nm. FIG. 6A-6C present the setting and measurements related to laser induced damage of the coating, according to some embodiments of the invention.

FIG. 6A shows the setting of the measurements in a measurement system 60. Samples 106 were mounted on an x-y-translational stage and illuminated by a Nd:YAG laser 61. For each irradiation, a new sample site was chosen. On-line damage detection was achieved with the help of a video microscope 64. The digital CCD camera of device 64 was operated by image analysis software, accomplishing subtraction of sample micrographs (before vs. after irradiation). According to ISO 11254, damage was defined as any detectable change of the sample morphology. The incident laser power was varied by changing the current of the pumping diodes; the power was measured by placing a calibrated power meter directly at the laser aperture. For the 1064 nm wavelength, the laser beam irradiated the sample directly. For the 532 nm wavelength, the beam had to be focused using a biconvex lens 62 (f=100 mm) in order to create visible damage on the samples (efficiency of ˜25% for second harmonic generation). The irradiation parameters were as follows: Wavelengths: 1064 nm, 532 nm; Pulse length: 1 μs; Repetition rate: 20 kHz; Beam size A_{eff} on sample: 1.8*10⁻² cm² (1064 nm), 3.9*10⁻⁴ cm² (532 nm); Max. intensity: 800 W/cm² (1064 nm), 8000 W/cm² (532 nm); Irradiation period: 15, 20, 30, 60, 120 s.

The laser beam profile on the sample surface for 1064 nm was recorded by direct exposure of a CCD camera (0°). Profiles were measured by means of pixel counts to have a diameter of ca. 1 mm for peak intensity and a diameter of ca. 2 mm for low intensity (30% the counts from peak). The beam profile for 532 nm was recorded by irradiating a white paper positioned at the sample surface plane, monitoring the stray light with the video microscope used for damage detection (sight angle of camera 60°). Profiles were measured by means of pixel counts to have a diameter of ca. 0.1 mm for peak intensity and a diameter of ca. 0.2 mm for low intensity (30% the counts from peak) due to focusing by lens 62.

For each laser intensity and irradiation period, sample 106 was irradiated at a new site. After irradiation, the extent of damage was assessed by visual inspection and with the help of video microscope 64 using digital image processing. In case of all samples (Type I and Type II) laser damage can be identified by the occurrence of white spots (increased light scattering, decreased absorbance. FIG. 6B illustrates the micro-morphology of the damaged sample sites, as investigated by SEM microscopy. The left panel shows that laser damage 66 results from a removal of coating material. This can be attributed to a thermal interaction process: the laser radiation is completely absorbed and converted into heat, which results in a high temperature on the sample (as observed by touching the rear side right after irradiation).

For on-line damage threshold determination, video micrographs of the test sites were taken before and after each exposure and then subtracted from each other. In case of no damage, no differences between the images were found. A typical damage event was characterized by clear differences in a spot having a diameter of ca. 1 mm.

The samples were irradiated at various laser intensities. After each exposure, the occurrence of damage was assessed as LIDT which is the average of the minimum intensity at which damage occurred and the maximum intensity at which no damage occurred. FIG. 6C illustrates damage measurements for determining LIDT at a test of subjecting sample Type I to 15 seconds exposure at 1064 nm. The measured LIDT was 746 W/cm²+/−0.8%. Next, this sample was exposed for 20, 30, 60 and 120 seconds. The resulting LIDT values were, correspondingly, 714, 589, 501 and 508 W/cm². For shorter exposure times, higher intensities were required to create damage on the exposed site. The similar LIDT data for 60 s and 120 s indicate a saturation level of ˜0.500 W/cm². This is the intensity at which an equilibrium temperature is reached, below which no damage is created.

In the case of Type II samples, the contrast between the damaged and undamaged areas was much lower. Initial irradiation with increasing exposure times (15 s, 20 s, 30 s) gave no indication of damage. The first damage was observed after 60 s irradiation and after that even with 15 s damages occurred. This might be explained by an incubation effect due to heating. The resulting LIDT values (after the incubation) were for exposure periods of 15, 20, 30 and 60 seconds, resulting LIDT values of, correspondingly, 435, 367, 390 and 449 W/cm². In contrast to Type I samples, the LIDT values of Type II samples did not show a significant exposure time dependence. The average value of ˜400 W/cm² is considerably lower than that of Type I samples (˜500 W/cm²). In another sample Type II, no damage at all could be detected even at maximum intensity. This can be attributed to the substrate thickness: In contrast to the former two samples (thickness=0.65 mm), the substrate of this Type II sample had a thickness of 3 mm Without wishing to be bound by theory, the heat flow from the exposed site to cooler regions of the substrate is believed to be much more effective. Thus substrate thickness may be designed to control heat dissipation and sensitivity to laser ablation of coating 120 in utensil 100.

The Type I sample was tested at 30 s and at 60 s exposure time with 532 nm illumination. The corresponding LIDT values were 1513 and 782 W/cm2. The 532 nm LIDT data are considerably higher compared to those obtained at 1064 nm. This can be attributed to the smaller spot size on the sample, due to the focusing lens used for the 532 nm tests. The first Type II sample was tested at 30 s and at 60 s exposure time to yield LIDT values of 910 and 564 W/cm2. Similar to 1064 nm, the LIDT data are lower for this Type II sample than for the Type I sample. For the other sample Type II sample, no damage could be detected at all, even at maximum intensity, similarly to the 1064 nm test. Table 10 summarizes the LIDT measurements with respect to these and additional tests.

TABLE 10
Summary of the Laser-induced damage threshold (LIDT) measurements

| Illumination | coating | substrate | LIDT |
|---|---|---|---|
| 193 nm, pulsed | Type I | SS-0.65 mm | 9500 KW/cm², 0.096 J/cm² |
| (10 ns) | Type II | SS-0.65 mm | 8000 KW/cm², 0.081 J/cm² |
| | | Al-3 mm | 8000 KW/cm², 0.086 J/cm² |
| 532 nm, 1064 nm, | Type I | SS-0.65 mm | 30 s-600 W/cm², |
| | | | 60 s-500 W/cm² |
| quasi CW | Type II | SS-0.65 mm | 30 s-400 W/cm², |
| | | | 60 s-400 W/cm² |
| | | Al-3 mm | Exceeds 800 W/cm² |
| 10600 nm CW | Type II | | Exceeds 3000 W/cm² |
| EUV - ca. 13 nm | Type I | | 1-on-1: >3.5 J/cm² |
| | | | 10-on-1: slightly below |
| | | | 3.5 J/cm² |

To summarize, LIDT values for sample Type I and Type II were measured at the wavelengths 1064 nm and 532 nm for different exposure times. Damage could be attributed to partial material removal associated with increased light scattering. The origin of laser damage is a thermal interaction process, indicated by the fact that for thick substrates (3 mm), no damage could be created due to better thermal relaxation. The LIDT of Type I sample was higher compared to Type II sample at both wavelengths; Type I sample showed larger exposure time dependence. The damage assessment for sample Type II was more difficult compared to sample Type I. For all samples, the contrast between the damaged sites and unexposed surroundings was rather faint and hardly visible under standard daylight illumination.

In certain embodiments, coating 120 may be laser ablated to reach very high resolution of ablation patterns, such as channels or cells of utensil 100. Advantageously, the inorganic nature of coating 120 enhances its stability with respect to organic materials used in other products (e.g., nitrocellulose). The enhanced stability allows forming thinner channels and cells within coatings 120 and thus improve respective utensil 100. The enhanced stability of coating 120 further allows standard treatments such as high temperature sterilization, e.g., in an autoclave, to be carried out without damaging coating 120 and/or the pattern thereof. Inorganic coating 120 further exhibits enhanced mechanical resistance to wear which further enhances the usability of utensils 100. The mechanical resistance also enables production procedures such as embossing which may not be applicable to less resistant coatings.

### Example 11

### Production of Embossed Foil

A substrate of aluminum foil was coated as described for Example 1, and then the foil was subjected to embossing in a regular pattern, to generate embossed foil 100 (FIG. 1D) suitable for use as a multi-well plate. In other embodiments, foil 100 is used as a backing for a bottomless multi-well plate (FIG. 1F).

### Example 12

### Production of Coated Multi-Well Plates

Aluminum is evaporated onto at least the inner well surfaces of multi-well plates 100 (FIG. 1A) made of a polymer or a similar substance by thermal resistive evaporation, electron beam evaporation, electric arc deposition, laser deposition, sputtering, or a similar technique, under an anhydrous atmosphere of nitrogen at a pressure of between 2×10⁻³ and 5×10⁻³ torr, and oxygen at a pressure between 2×10⁻⁴ torr and 5×10⁻⁴ torr. In other experiments, a 20% higher oxygen concentration is utilized. The coating is applied at a thickness between 30-250 microns. Thus, coated plates are produced, having an Al/Al₂O₃ layer deposited on the inner well surfaces.

### Example 13

### Linkage of Various Functional Ligands to Coated Surfaces

In certain embodiments, utensil 100 may be configured to enhance binding of specified molecules. The coated samples (foil, plates or any other embodiments of utensil 100) are cleaned, e.g. using isopropyl alcohol, ethanol, or a similar reagent. After baking at 110° C. for 3 h, slides are treated with a 2% (v/v) solution of N-(trimethoxysilylpropyl)-N,N,N-trimethylammonium chloride (50% in methanol; available from United Chemical Technologies, Inc.) in dry, HPLC-grade toluene for 45 min, followed by extensive rinsing with toluene and air-drying. Once this stage is reached, a variety of ligand attachment schemes, all involving incubations at room temperature, are possible. Hemin (ferriprotoporphyrin 1×) can be covalently attached by overnight incubation of the silanized slides with a toluene solution saturated with the N-hydroxysuccinamide diester of hemin, followed by washing with toluene and a air-drying. Alternatively, the slides can be treated with a solution of 50% glutaraldehyde (v/v) in phosphate buffer (pH 8.0) for 30 min, rinsed with copious amounts of water, allowed to dry, and then a variety of amine-containing ligands can be attached by overnight incubation. For Example, an overnight incubation with a 2% (w/w) solution of desferrioxamine B, followed by a water rinse, and then a 2-min incubation with 10 mM ferrous sulfate (pH 7.0), another rinse with water and air drying, produces slides containing the attached siderophore ferrioxamine B.

A schematic non-limiting example of the process for coating surfaces comprises the following three steps: (i) linking a silane-based molecule to the surface; (ii) linking a cross-linker to the ending of the silane; and (iii) attaching the bio-molecule to the terminal of the cross linker molecule.

### Example 14

### Use of Laser Ablation to Create Coated Plates with Hydrophobic and Hydrophilic Regions

Laser ablation may be used to add pattering, e.g. pattering of hydrophobic and hydrophilic regions, to the described coatings via a dry, non-contact, digital, single step process. Fiber and Ultraviolet (UV) lasers are particularly ideal for ablation, because metalized materials can effectively be removed with minimal damage to the carrier substrate. The process can be completed on either side of a material. The laser source and wavelength used is dependent upon the material to be processed, including the type of coating to be removed and the type of substrate below the coating. Those skilled in the art will appreciate in light of the present disclosure that the proper choice of laser wavelength also depends on the required ablation width and quality. For Example, the majority of the described coatings ablate very well with UV laser wavelengths.

As another Example, CO₂ lasers alone may not be suitable for removing conductive coatings from polymers. This is because generally polymers absorb CO₂ laser wavelengths, causing damage to the substrate layer under the conductive coating. The polymer underneath can also be damaged from the heat generated during the conductive coating's vaporization. However, CO₂ lasers can be used in conjunction with Fiber, YAG, and UV lasers for these types of purposes.

In certain embodiments, coating 120 is configured to enhance binding of specified molecules.

In certain embodiments, coating 120 is configured to exhibit a fluorescence rate of 10⁻⁹ or less of a number of incoming photons, over a range of incoming wavelengths of 200-280 nm and a range of fluorescence wavelengths of 280 nm-380 nm.

In certain embodiments, coating 120 is configured to exhibit hemispherical reflectance of less than 2% over a range of wavelengths ranging from 0.1-10 μm.

In certain embodiments, coating 120 is configured to exhibit a surface porosity of 50%, with a majority of the pores between 5-20 microns in diameter.

In certain embodiments, coating 120 is configured to exhibit no significant cytotoxicity as measured using L929 mouse fibroblasts in culture medium containing 10% fetal calf serum, according to DIN EN ISO 10993-5.

In certain embodiments, coating 120 is configured to exhibit a laser-induced damage threshold of 400 W/cm² or higher under conditions of a direct irradiation for 60 seconds at a wavelength of 1064 nanometers (nm), a pulse length of 1 μs, a repetition rate of 20 kHz, and a beam size (A_{eff}) on the sample of 1.8*10⁻² cm².

In certain embodiments, coating 120 is conjugated to a polypeptide, a protein, an oligonucleotide, or streptavidin.

### Hydrophilicity and Binding Control

Tests were carried out for several types of coatings 120 with respect to the characteristics: hydrophilicity, binding to biological molecules and fluorescent background radiation. Coatings 120 exhibited very high levels of hydrophilicity, which are appropriate for handling biological and many chemical samples. The inventors discovered that coatings 120 exhibited good binding characteristics to biological molecules.

For example, as illustrated in FIG. 5H for patterned coating 120 in form of dots on a glass surface, both spotting with fluorescent protein (top five rows) and spotting with labeled DNA (bottom five rows) yielded ordered dots (diagram on the left). After washing the glass slides, the spots retained their form and fluorescent measurements (diagram on the right).

Regarding the experimental details, the illustrated configuration included coating 120 on glass slides. Coating 120 included porous layer 122 with over-coating layer 121 comprising epoxy-terminated silane for controlling spot size. The bound molecules 125 were fluorescently labelled protein (green, Rabbit anti mouse IgG labeled Alexa 555, Concentration=0.2 mg/ml in buffer) and DNA (red, oligo-labeled with ATTO 647 dye, Concentration=1 μm in buffer II). The protein and DNA solutions were printed upon the coated glass slides with an inkjet printer, having drop volume of 350 pl (1drop/spot), at the illustrated 5×10 array for each of the protein and the DNA. Immobilization was carried out overnight, at room temperature and 70% humidity. The non-linked molecules were washed with suitable washing buffers (wash with buffer 1 for 10 min, wash with buffer 2 for 5 min—repeated twice), rinsed with water and were dried in centrifuge. The read out of fluorescence signal from linked molecules and background was examined after spotting and after washing steps. Scanning was performed at 10 μm/pixel, Brightness=50, Contrast=50.

The spots are dot shaped with homogenous morphology, no spreading or “shadowing” effect on peripheral of the spot, very good signal intensity from spot and relatively low background intensity The experiment illustrates the highly effective binding of biological molecules by coating 120.

In the above description, an embodiment is an Example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in certain embodiments other than the ones outlined in the description above.

The invention is not limited to those diagrams or to the corresponding descriptions. For Example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method comprising reducing background fluorescence in a utensil by producing at least an imaged region of the utensil to have a coating comprising at least one porous fractal ceramic layer produced by reactive vapor deposition and comprising at least one oxide of: aluminum, titanium, tantalum, niobium, zirconium, silicon, thorium, cadmium and tungsten, wherein the coating is configured to exhibit hemispherical reflectance of less than 2% over a range of wavelengths ranging from 0.1-10 μm.

2. (canceled)

3. (canceled)

4. The method of claim 1, further comprising designing a specified pattern with respect to a specified hydrophobic/hydrophilic character thereof and according to a microscopy target, and depositing the coating upon at least the imaged region of the utensil, according to the designed specified pattern.

5. (canceled)

6. (canceled)

7. The method of claim 4, further comprising producing the patterned coating by laser ablation of the coating.

8. The method of claim 4, wherein the imaged region comprises at least one of: a well bottom in a multi well plate, a foil or regions thereof, an embossed foil or regions thereof, a continuous array tape, a foil metal insert, a lab-on-a-foil, DNA or protein microarrays, a region on a glass slide, a flow channel in a flow cell, a microscope pad or regions thereof, a microbead, a membrane or regions thereof and a biochip.

9. (canceled)

10. (canceled)

11. (canceled)

12. (canceled)

13. (canceled)

14. The method of claim 1, further comprising configuring the coating to enhance binding of specified molecules.

15. (canceled)

16. The method of claim 1, further comprising configuring the coating to have a cytotoxicity below a specified threshold.

17. The method of claim 1, further comprising configuring the coating to have an outgassing level below a specified threshold.

18. (canceled)

19. The method of claim 1, further comprising enhancing marker or binding resolution by adjusting a porosity of the coating.

20. A utensil having a coating comprising at least one porous fractal ceramic layer produced by reactive vapor deposition and comprising at least one oxide of: aluminum, titanium, tantalum, niobium, zirconium, silicon, thorium, cadmium and tungsten, wherein the coating is configured to exhibit hemispherical reflectance of less than 2% over a range of wavelengths ranging from 0.1-10 μm to reduce background fluorescence of at least one imaged region of the utensil.

21. (canceled)

22. (canceled)

23. The utensil of claim 20, wherein the utensil is a multi-well plate and the at least one imaged region comprises well bottoms thereof.

24. The utensil of claim 20, wherein the utensil is a glass slide and the at least one imaged region is patterned thereon.

25. The utensil of claim 20, wherein the utensil is a flow cell and the at least one imaged region comprises at least a part of a flow channel therein.

26. The utensil of claim 20, wherein the utensil is a foil having the coating.

27. The utensil of claim 20, wherein the utensil is an embossed foil having coated indentations as imaged regions.

28. The utensil of claim 20, wherein the utensil comprises a foil having the coating and attached to the utensil at the at least one imaged region.

29. The utensil of claim 20, wherein the utensil is a membrane having the coating on both sides.

30. (canceled)

31. The utensil of claim 20, wherein the coating is configured to exhibit a fluorescence rate of 10⁻⁹ or less of a number of incoming photons, over a range of incoming wavelengths of 200-280 nm and a range of fluorescence wavelengths of 280 nm-380 nm.

32. The utensil of claim 20, wherein the coating is configured to exhibit at least one of:
a hemispherical reflectance of less than 2% over a range of wavelengths ranging from 0.1-10 μm,
a surface porosity of 50%, with a majority of the pores between 5-20 microns in diameter,
no significant cytotoxicity as measured using L929 mouse fibroblasts in culture medium containing 10% fetal calf serum, according to DIN EN ISO 10993-5, and
a laser-induced damage threshold of 400 W/cm² or higher under conditions of a direct irradiation for 60 seconds at a wavelength of 1064 nanometers (nm), a pulse length of 1 μs, a repetition rate of 20 kHz, and a beam size (A_{eff}) on the sample of 1.8·10⁻² cm².

33. (canceled)

34. (canceled)

35. (canceled)

36. The utensil of claim 20, wherein the coating is conjugated to a polypeptide, a protein, an oligonucleotide, or streptavidin.

37. (canceled)

38. (canceled)

39. (canceled)

40. (canceled)

41. The utensil of claim 20, wherein the coating has an atomic ratio of aluminum to oxygen atoms which is between 0.66-4.8.
